(19) United States
(12) Patent Application Publication
(10) Pub. No.: US 2005/0054097 A1
(43) Pub. Date: Mar. 10, 2005

(54) EX-VIVO expansion of hematopoietic system cell populations in mononuclear cell cultures

(76) Inventors: Tony Peled, Mevaseret Zion (IL); Avi Treves, Mevaseret Zion (IL); Oren Rosen, Jerusalem (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/767,064

(22) Filed: Jan. 29, 2004

Related U.S. Application Data
(60) Provisional application No. 60/452,545, filed on Mar. 7, 2003.

(30) Foreign Application Priority Data
Nov. 17, 2002 (IL) 152904

(51) Int. Cl. C12N 5/08

(52) U.S. Cl. 435/372; 424/93.7

(57) ABSTRACT

Ex-vivo methods of expanding hematopoietic stem cells of hematopoietic mononuclear cells that comprise a major fraction of hematopoietic committed cells and a minor fraction of hematopoietic stem and progenitor cells, expanded populations of hematopoietic stem cells obtained thereby and their uses are disclosed.

### FIELD AND BACKGROUND OF THE INVENTION

The present invention relates to methods of ex-vivo expansion (self-renewal) of hematopoietic stem cells present in the hematopoietic mononuclear cells fraction of a blood sample and to expanded (self-renewed) populations of hematopoietic stem cells obtained thereby. The present invention further relates to therapeutic applications in which these methods and/or the expanded hematopoietic stem cell populations obtained thereby are utilized.

An increasing need for ex-vivo cultures of hematopoietic stem cells has arisen, in particular for purposes such as stem cell expansion and retroviral-mediated gene transduction. Methods for generating ex-vivo cultures of stem cells, however, typically result in a rapid decline in stem cell population activity, further resulting in a markedly impaired self-renewal potential and diminished transplantability of the cultured cell populations. The need to improve such methods is widely acknowledged. Additionally, applications in gene therapy using retroviral vectors necessitate the use of proliferating hematopoietic stem cells, yet require that these cells remain undifferentiated while in culture, in order to maintain long-term expression of the transduced gene. Thus, the ability to maintain ex-vivo cultures of hematopoietic stem cell populations with long-term, self-renewal capacity is of critical importance for a wide array of medical therapeutic applications.

Presently, expansion of renewable stem cells have been achieved either by growing the stem cells over a feeder layer of fibroblast cells, or by growing the cells in the presence of the early acting cytokines thrombopoietin (TPO), interleukin-6 (IL-6), an FLT-3 ligand and stem cell factor (SCF) (Madlambayan G J et al. (2001) J Hematother Stem Cell Res 10: 481, Punzel M et al. (1999) Leukemia 13: 92, and Lange W et al. (1996) Leukemia 10: 943). While expanding stem cells over a feeder layer results in vast, substantially endless cell expansion, expanding stem cells without a feeder layer, in the presence of the early acting cytokines listed able, results in an elevated degree of differentiation (see controls described in the Examples section and Leslie NR et al. (Blood (1998) 92: 4798), Petzer A L et al. (1996) J Exp Med Jun 183: 2551, Kawa Y et al. (2000) Pigment Cell Res 8: 73).

Hence, self-renewal (expansion) of hemopoietic stem and progenitor cells, both in vivo and in vitro, is limited by cell differentiation. Differentiation in the hematopoietic system involves, among other changes, altered expression of surface antigens (Sieff C, Bicknell D, Caine G, Robinson J, Lam G, Greaves MF (1982) Changes in cell surface antigen expression during hematopoietic differentiation. Blood 60:703). In normal human, most of the hematopoietic pluripotent stem cells and the lineage committed progenitor cells are CD34+. The majority of cells are CD34+CD38+, with aminority of cells (<10%) being CD34+CD38−. The CD34+CD38− phenotype appears to identify the most immature hematopoietic cells, which are capable of self-renewal and multilineage differentiation. The CD34+CD38− cell fraction contains more long-term culture initiating cells (LTC-IC) pre-CFU and exhibits longer maintenance of their phenotype and delayed proliferative response to cytokines as compared with CD34+CD38+cells. CD34+CD38− cells can give rise to lymphoid and myeloid cells in vitro and have an enhanced capacity to repopulate SCID mice (Bhatia M, Wang J C Y, Kapp U, Bonnet D, Dick J E (1997) Purification of primitive human hematopoietic cells capable of repopulating immune-deficient mice. Proc Natl Acad Sci USA 94:5320). Moreover, in patients who received autologous blood cell transplantation, the number of CD34+CD38− cells infused correlates positively with the speed of hematopoietic recovery. In line with these functional features, CD34+CD38− cells have been shown to have detectable levels of telomerase.

The presently published works on ex-vivo expansion of hematopoietic stem and progenitor cells involve starting inoculums of cells, which are highly enriched with progenitor cells that express CD34 or, the even earlier, AC133 antigens [Dexter, T. M., T. D. Allen, and L. G. Lajtha, Conditions controlling the proliferation of haemopoietic stem cells in vitro. J.Cell Physiol, 1977. 91(3): p. 335-44; Muench, M. O., J. G. Schneider, and M. A. Moore, Interactions among colony-stimulating factors, IL-1 beta, IL-6, and kit-ligand in the regulation of primitive murine hematopoietic cells. Exp. Hematol., 1992. 20(3): p. 339-49; Verfaillie, C. M., Direct contact between human primitive hematopoietic progenitors and bone marrow stroma is not required for long-term in vitro hematopoiesis. Blood, 1992. 79(11): p. 2821-26; Migliaccio, G., A. R. Migliaccio, M. L. Druzin, P. J. Giardina, K. M. Zsebo, and J. W. Adamson, Long-term generation of colony-forming cells in liquid culture of CD34+cord blood cells in the presence of recombinant human stem cell factor. Blood, 1992. 79(10): p. 2620-27; Purdy, M. H., C. J. Hogan, L. Hami, 1. McNiece, W. Franklin, R. B. Jones, S. I. Bearman, R. J. Berenson, P. J. Cagnoni, and S. Heimfeld, Large volume ex-vivo expansion of CD34-positive hematopoietic progenitor cells for transplantation. J. Hematother., 1995. 4(6): p. 515-25; McNiece, I., R. Andrews, M. Stewart, S. Clark, T. Boone, and P. Quesenberry, Action of interleukin-3, G-CSF, and GM-CSF on highly enriched human hematopoietic progenitor cells: synergistic interaction of GM-CSF plus G-CSF. Blood, 1989. 74(1): p. 110-14; Colter, M., M. Jones, and S. Heimfeld, CD34+progenitor cell selection: clinical transplantation, tumor cell purging, gene therapy, ex-vivo expansion, and cord blood processing. J Hematother, 1996. 5(2): p. 179-84; Kohler, T., R. Plettig, W. Wetzstein, B. Schaffer, R. Ordemann, H. O. Nagels, G. Ehninger, and M. Bornhauser, Defining optimum conditions for the ex-vivo expansion of human umbilical cord blood cells. Influences of progenitor enrichment, interference with feeder layers, early-acting cytokines and agitation of culture vessels. Stem Cells, 1999. 17(1): p. 19-24].

As it was shown that initiation of ex-vivo cultures with the entire mononuclear cells (MNC) fraction in the presence of cytokines led to expansion of CFUc during the first weeks of culturing, followed by a rapid deterioration of the cultures, it has been widely accepted heretofore that purification of CD34+ (or AC 133) cells is a prerequisite for achieving successful ex-vivo expansion of hematopoietic stem cells [Briddell, R., Keren, B. P., Zilm, K. L., et al. Purification of CD34+ cell is essential for optimal ex-vivo expansion of umbilical cord blood cells. J. Hematother. 6:145, 1997; Ian K McNiece, Gregory B. Stoney, Brent P. Keren, and Robert A. Briddell CD34+cell selection from frozen cord blood products using Isolex 300i and cliniMACS™ selection device. Journal of hematotherapy 7:457-461 (1998)] as well as long-term culture colony forming cells (LTC-CFUc).

WO 99/40783, WO 00/18885 and Peled et al, Brit. J. Haematol. 116:655 2002, all of which are incorporated by reference as if fully set forth herein, teach the effect of free copper present in cells on the modulation of the balance between self-renewal and differentiation of hematopoietic progenitor cells. These references teach that the addition of agents that are capable of reducing the cell copper content, along with early acting cytokines, to CD34+ cell cultures results in long term CD34+ cell expansion ex-vivo in culture. According to teachings of these references, such agents preferably include transition metal chelators that are capable of binding copper, such as, for example, linear polyamines (e.g., tetraethylenepentamine, TEPA). Hence, it is shown in these references that the addition of 5-10 μM TEPA to CD34+cell cultures in the presence of early acting cytokines reduced cell copper content by 30% (as measured by atomic absorption), and extended the duration of the long-term cultures in terms of long-term CFU and CD34+ cell expansion.

However, the methods disclosed in these references also involve purification of stem or progenitor cells prior to their expansion in cultures.

Thus, using present day technology, stem cells cannot be expanded unless first substantially enriched or isolated to homogeneity and therefore the presently known methods of ex-vivo expanding stem cell populations are limited by the laborious and costly process of stem cells enrichment prior to initiation of cultures.

There is thus a widely recognized need for, and it would be highly advantageous to have, methods of ex-vivo expanding hematopoietic stem cells without prior stem cells enrichment.

### SUMMARY OF THE INVENTION

The present invention discloses the use of various agents in expanding hematopoietic stem cells present in the hematopoietic mononuclear cells fraction of a blood sample, without the use of a prior stem cells enrichment procedure, to expanded (self-renewed) populations of hematopoietic stem cells obtained thereby and to their uses.

According to one aspect of the present invention there is provided a method of ex-vivo expanding a population of hematopoietic stem cells, while at the same time, substantially inhibiting differentiation of the hematopoietic stem cells ex-vivo.

In one embodiment, the method comprises providing hematopoietic mononuclear cells which comprise a major fraction of hematopoietic committed cells and a minor fraction of hematopoietic stem and progenitor cells, with ex-vivo culture conditions for ex-vivo cell proliferation and, at the same time, for reducing an expression and/or activity of CD38, thereby expanding a population of the hematopoietic stem cells, while at the same time, substantially inhibiting differentiation of the hematopoietic stem cells ex-vivo.

In another embodiment the method comprises providing the hematopoietic mononuclear cells with ex-vivo culture conditions for ex-vivo cell proliferation and, at the same time, for reducing a capacity of the hematopoietic mononuclear cells in responding to retinoic acid, retinoids and/or Vitamin D, thereby expanding the population of the hematopoietic stem cells while at the same time, substantially inhibiting differentiation of the stem cells ex-vivo.

In still another embodiment the method comprises providing the hematopoietic mononuclear cells with ex-vivo culture conditions for ex-vivo cell proliferation and, at the same time, for reducing a capacity of the hematopoietic mononuclear cells in responding to signaling pathways involving the retinoic acid receptor, retinoic-X receptor and/or Vitamin D receptor, thereby expanding the population of the hematopoietic stem cells while at the same time, substantially inhibiting differentiation of the hematopoietic stem cells ex-vivo.

In yet another embodiment the method comprises providing the hematopoietic mononuclear cells with ex-vivo culture conditions for ex-vivo cell proliferation and at the same time, for reducing a capacity of the hematopoietic mononuclear cells in responding to signaling pathways involving PI 3-kinase, thereby expanding the population of the hematopoietic stem cells while at the same time, substantially inhibiting differentiation of the hematopoietic stem cells ex-vivo.

In still another embodiment, the method comprises providing the hematopoietic mononuclear cells with ex-vivo culture conditions for ex-vivo cell proliferation and, at the same time, with nicotinamide, a nicotinamide analog, a nicotinamide or a nicotinamide analog derivative or a nicotinamide or a nicotinamide analog metabolite, thereby expanding the population of the hematopoietic stem cells while at the same time, substantially inhibiting differentiation of the hematopoietic stem cells ex-vivo.

In yet another embodiment, the method comprises providing the hematopoietic mononuclear cells with ex-vivo culture conditions for ex-vivo cell proliferation and, at the same time, with a PI 3-kinase inhibitor, thereby expanding the population of the hematopoietic stem cells while at the same time, substantially inhibiting differentiation of the hematopoietic stem cells ex-vivo.

In still another embodiment, the method comprises providing the hematopoietic mononuclear cells with ex-vivo culture conditions for ex-vivo cell proliferation and, at the same time, with one or more copper chelator(s) or copper chelate(s), thereby expanding the population of the hematopoietic stem cells while at the same time, substantially inhibiting differentiation of the hematopoietic stem cells ex-vivo.

Further according to the present invention, there are provided ex-vivo expanded populations of hematopoietic stem cells, obtained by the methods described hereinabove.

According to another aspect of the present invention there is provided a method of hematopoietic cells transplantation or implantation.

In one embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and, at the same time, for reducing an expression and/or activity of CD38, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In another embodiment, the method comprises (a) obtaining hematopoietic mononuclear cells which comprise a major fraction of hematopoietic committed cells and a minor fraction of hematopoietic stem and progenitor cells from a donor (b) providing the hematopoietic mononuclear cells with ex-vivo culture conditions for cell proliferation and, at the same time, for reducing a capacity of the hematopoietic mononuclear cells in responding to retinoic acid, retinoids and/or Vitamin D, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In yet another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and, at the same time, for reducing a capacity of the hematopoietic mononuclear cells in responding to responding to signaling pathways involving the retinoic acid receptor, the retinoid X receptor and/or the Vitamin D receptor, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In still another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and, at the same time, for reducing a capacity of the hematopoietic mononuclear cells in responding to responding to signaling pathways involving PI-3 kinase, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In yet another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and with nicotinamide, a nicotinamide analog, a nicotinamide or a nicotinamide analog derivative or a nicotinamide or a nicotinamide analog metabolite, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In still yet another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and with a PI 3-kinase inhibitor, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In yet another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and, at the same time, for reducing the expression and/or activity of PI 3-kinase, thereby expanding a population of the hematopoietic stem cells, while at the same time, substantially inhibiting differentiation of the hematopoietic stem cells ex-vivo; and (c) transplanting or implanting the hematopoietic stem cells to a recipient.

In still yet another embodiment, the method comprises (a) obtaining hematopoietic mononuclear cells which comprise a major fraction of hematopoietic committed cells and a minor fraction of hematopoietic stem and progenitor cells from a donor; (b) providing the hematopoietic mononuclear cells with ex-vivo culture conditions for cell proliferation and with one or more copper chelator(s) or chelator(s), thereby expanding a population of the hematopoietic stem cells, while at the same time, substantially inhibiting differentiation of the hematopoietic stem cells ex-vivo; and (c) transplanting or implanting the hematopoietic stem cells to a recipient.

The donor and the recipient in the methods above can be a single individual or different individuals, for example, allogeneic or xenogeneic individuals.

According to still another aspect of the present invention there are provided transplantable hematopoietic cell preparations.

In one embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent for reducing an expression and/or activity of CD38, while at the same time, substantially inhibiting differentiation of said hematopoietic stem cells, and a pharmaceutically acceptable carrier.

In another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent for reducing an expression and/or activity of PI 3-kinase, while at the same time, substantially inhibiting differentiation of said hematopoietic stem cells, and a pharmaceutically acceptable carrier.

In still another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent, the agent reducing a capacity of the hematopoietic mononuclear cells in responding to retinoic acid, retinoids and/or Vitamin D, while at the same time, substantially inhibiting differentiation of said hematopoietic stem cells, and a pharmaceutically acceptable carrier.

In yet another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent, the agent reducing a capacity of the hematopoietic mononuclear cells in responding to retinoic acid receptor, retinoid X receptor and/or Vitamin D receptor signaling, while at the same time, substantially inhibiting differentiation of said hematopoietic stem cells, and a pharmaceutically acceptable carrier.

In still another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent, the agent reducing a capacity of the hematopoietic mononuclear cells in responding to PI 3-kinase signaling, while at the same time, substantially inhibiting differentiation of said hematopoietic stem cells, and a pharmaceutically acceptable carrier.

In yet another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent selected from the group consisting of nicotinamide, a nicotinamide analog, a nicotinamide or a nicotinamide analog derivative and a nicotinamide or a nicotinamide analog metabolite, while at the same time, substantially inhibiting differentiation of said hematopoietic stem cells, and a pharmaceutically acceptable carrier.

In still another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of a PI 3-kinase inhibitor, while at the same time, substantially inhibiting differentiation of said hematopoietic stem cells, and a pharmaceutically acceptable carrier.

In yet another embodiment, a transplantable hematopoietic cell preparation of the present invention comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of one or more copper chelator(s) or copper chelate(s), while at the same time, substantially inhibiting differentiation of said hematopoietic stem cells, and a pharmaceutically acceptable carrier.

According to an additional aspect of the present invention there is provide a method of adoptive immunotherapy.

In one embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and, at the same time, for reducing an expression and/or activity of CD38, thereby expanding a population of the hematopoietic stem cells, while at the same time, substantially inhibiting differentiation of the hematopoietic stem cells; and (c) transplanting said hematopoietic stem cells to the recipient.

In another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and, at the same time, for reducing a capacity of the hematopoietic mononuclear cells in responding to retinoic acid, retinoids and/or Vitamin D, thereby expanding a population of the stem cells, thereby expanding a population of the hematopoietic stem cells, while at the same time, substantially inhibiting differentiation of the hematopoietic stem cells; and (c) transplanting said hematopoietic stem cells to the recipient.

In still another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and, at the same time, for reducing a capacity of the hematopoietic mononuclear cells in responding to signaling pathways involving the retinoic acid receptor and/or the retinoid X receptor and/or the Vitamin D receptor, thereby expanding a population of the hematopoietic stem cells, while at the same time, substantially inhibiting differentiation of the hematopoietic stem cells; and (c) transplanting said hematopoietic stem cells to the recipient.

In yet another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and, at the same time, for reducing a capacity of the hematopoietic mononuclear cells in responding to signaling pathways involving PI 3-kinase, thereby expanding a population of the hematopoietic stem cells, while at the same time, substantially inhibiting differentiation of the hematopoietic stem cells; and (c) transplanting said hematopoietic stem cells to the recipient.

In still another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and with nicotinamide, a nicotinamide analog, a nicotinamide or a nicotinamide analog derivative or a nicotinamide or a nicotinamide analog metabolite, thereby expanding a population of the hematopoietic stem cells, while at the same time, substantially inhibiting differentiation of the hematopoietic stem cells; and (c) transplanting said hematopoietic stem cells to the recipient.

In yet another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and with a PI 3-kinase inhibitor, thereby expanding a population of the hematopoietic stem cells, while at the same time, substantially inhibiting differentiation of the hematopoietic stem cells; and (c) transplanting said hematopoietic stem cells to the recipient.

In still another embodiment the method comprises (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and with one or more copper chelator(s) or chelate(s), thereby expanding a population of the hematopoietic stem cells, while at the same time, substantially inhibiting differentiation of the hematopoietic stem cells; and (c) transplanting said hematopoietic stem cells to the recipient.

Further according to an aspect of the present invention, there is provided a method of genetically modifying stem cells with an exogene.

In one embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and, at the same time, for reducing an expression and/or activity of CD38, the by expanding a population of the hematopoietic stem cells, while at the same time, substantially inhibiting differentiation of the hematopoietic stem cells ex-vivo; and (c) genetically modifying said hematopoietic stem cells with the exogene.

In another embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and, at the same time, for reducing an expression and/or activity of PI 3-kinase, thereby expanding a population of the hematopoietic stem cells, while at the same time, substantially inhibiting differentiation of the hematopoietic stem cells ex-vivo; and (c) genetically modifying said hematopoietic stem cells with the exogene.

In still another embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and, at the same time, for reducing a capacity of the hematopoietic mononuclear cells in responding to retinoic acid, retinoids and/or Vitamin D, thereby expanding a population of the hematopoietic stem cells, while at the same time, substantially inhibiting differentiation of the hematopoietic stem cells ex-vivo; and (c) genetically modifying said hematopoietic stem cells with the exogene.

In yet another embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and, at the same time, for reducing a capacity of the hematopoietic mononuclear cells in responding to signaling pathways involving the retinoic acid receptor and/or the retinoid X receptor and/or the Vitamin D receptor, thereby expanding a population of the hematopoietic stem cells, while at the same time, substantially inhibiting differentiation of the hematopoietic stem cells ex-vivo; and (c) genetically modifying said hematopoietic stem cells with the exogene.

In still another embodiment, the method comprises (a) obtaining hematopoietic mononuclear cells which comprise a major fraction of hematopoietic committed cells and a minor fraction of hematopoietic stem and progenitor cells (b) providing the hematopoietic mononuclear cells with ex-vivo culture conditions for cell proliferation and, at the same time, for reducing a capacity of the hematopoietic mononuclear cells in responding to signaling pathways involving PI 3-kinase, thereby expanding a population of the hematopoietic stem cells, while at the same time, substantially inhibiting differentiation of the hematopoietic stem cells ex-vivo; and (c) genetically modifying said hematopoietic stem cells with the exogene.

In yet another embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and with nicotinamide, a nicotinamide analog, a nicotinamide or a nicotinamide analog derivative or a nicotinamide or a nicotinamide analog metabolite, thereby expanding a population of the hematopoietic stem cells, while at the same time, substantially inhibiting differentiation of the hematopoietic stem cells ex-vivo; and (c) genetically modifying said hematopoietic stem cells with the exogene.

In still another embodiment, the method comprises (a) obtaining hematopoietic mononuclear cells which comprise a major fraction of hematopoietic committed cells and a minor fraction of hematopoietic stem and progenitor cells (b) providing the hematopoietic mononuclear cells with ex-vivo culture conditions for cell proliferation and with a PI 3-kinase inhibitor, thereby expanding a population of the hematopoietic stem cells, while at the same time, substantially inhibiting differentiation of the hematopoietic stem cells ex-vivo; and (c) genetically modifying said hematopoietic stem cells with the exogene.

In yet another embodiment, the method comprises (a) obtaining hematopoietic mononuclear cells which comprise a major fraction of hematopoietic committed cells and a minor fraction of hematopoietic stem and progenitor cells; (b) providing the hematopoietic mononuclear cells with ex-vivo culture conditions for cell proliferation and with one or more copper chelator(s) or chelate(s), thereby expanding a population of the hematopoietic stem cells, while at the same time, substantially inhibiting differentiation of the hematopoietic stem cells ex-vivo; and (c) genetically modifying said hematopoietic stem cells with the exogene.

In a preferred embodiment, genetically modifying the cells is effected by a vector which comprises the exogene, which vector is, for example, a viral vector or a nucleic acid vector.

According to still a further aspect of the present invention there is provided a hematopoietic stem cells collection/culturing bag supplemented with an effective amount of a retinoic acid receptor antagonist, a retinoid X receptor antagonist and/or a Vitamin D receptor antagonist, with an effective amount of nicotinamide a nicotinamide analog, a nicotinamide or a nicotinamide analog derivative and a nicotinamide or a nicotinamide analog metabolite, with an effective amount of a PI 3-kinase inhibitor, or with an effective amount of a copper chelator or chelate, each of which substantially inhibits cell differentiation of a hematopoietic stem cells fraction of hematopoietic mononuclear cells which comprise a major fraction of hematopoietic committed cells and a minor fraction of hematopoietic stem and progenitor cells.

According to an additional aspect of the present invention, there is provided an assay of determining whether an agent/molecule is an effective hematopoietic stem cell expansion agent. The assay comprises culturing hematopoietic mononuclear cells which comprise a major fraction of hematopoietic committed cells and a minor fraction of hematopoietic stem and progenitor cells in the presence of tested agent/molecule and monitoring expansion of the hematopoietic stem cells, wherein if increased expansion and decreased differentiation of the hematopoietic stem cells occurs, as compared to non-treated hematopoietic mononuclear cells, the tested agent/molecule is an effective hematopoietic stem cell expansion agent.

The agent/molecule can be a retinoic acid receptor antagonist, a retinoid X receptor antagonist, a Vitamin D receptor antagonist, nicotinamide and an analog, a derivative and a metabolite thereof, a PI 3-kinase inhibitor, a copper chelator and a copper chelate.

According to further features in preferred embodiments of the invention described below, reducing the expression and/or activity of CD38 is effected by an agent that downregulates CD38 expression.

According to still further features in the described preferred embodiment. The agent that downregulates CD38 expression is selected from the group consisting of a retinoic acid receptor antagonist, a retinoid X receptor antagonist and a Vitamin D receptor antagonist. Alternatively, this agent is an antagonist for reducing a capacity of the stem cells in responding to retinoic acid, retinoid and/or Vitamin D. Further alternatively, the agent that downregulates CD38 expression is a PI 3-kinase inhibitor.

According to still further features in the described preferred embodiments the agent that downregulates CD38 expression is a polynucleotide.

According to still further features in the described preferred embodiments the polynucleotide encodes an anti CD38, an anti retinoic acid receptor, an anti retinoid X receptor, an anti Vitamin D receptor or an anti PI 3-kinase antibody or intracellular antibody.

According to still further features in the described preferred embodiments the polynucleotide is a small interfering polynucleotide molecule directed to cause intracellular CD38, retinoic acid receptor, retinoid X receptor, Vitamin D receptor or PI 3-kinase mRNA degradation.

According to still further features in the described preferred embodiments the small interfering polynucleotide molecule is selected from the group consisting of an RNAi molecule, an anti-sense molecule, a rybozyme molecule and a DNAzyme molecule.

According to further features in preferred embodiments of the invention described below, reducing the expression and/or activity of CD38 is effected by an agent that inhibits CD38 activity. The agent can be, for example, nicotinamide, a nicotinamide analog, a nicotinamide or a nicotinamide analog derivative or a nicotinamide or a nicotinamide analog metabolite. The nicotinamide analog is preferably selected from the group consisting of benzamide, nicotinethioamide, nicotinic acid and α-amino-3-indolepropionic acid.

According to further features in preferred embodiments of the invention described below, reducing the expression and/or activity of CD38 is effected by an agent that inhibits PI 3-kinase activity.

According to further features in preferred embodiments of the invention described below, providing the stem cells with the conditions for ex-vivo cell proliferation comprises providing the cells with nutrients and with cytokines.

According to still further features in the described preferred embodiment the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin.

According to still further features in the described preferred embodiments the cytokines are late acting cytokines, such as, but not limited to, granulocyte colony stimulating factor, granulocyte/macrophage colony stimulating factor, erythropoietin, FGF, EGF, NGF, VEGF, LIF, Hepatocyte growth factor and macrophage colony stimulating factor.

According to still further features in the described preferred embodiments the hematopoietic mononuclear cells are derived from a source selected from the group consisting of bone marrow, peripheral blood and neonatal umbilical cord blood.

According to still further features in the described preferred embodiments reducing the capacity of the hematopoietic mononuclear cells in responding to signaling pathways is reversible, e.g., inherently reversible.

According to still further features in the described preferred embodiments reducing the capacity of the hematopoietic mononuclear cells in responding to the above antagonists and/or signaling pathways of the above receptors is by ex-vivo culturing the hematopoietic mononuclear cells in a presence of an effective amount of at least one retinoic acid receptor antagonist, at least one retinoid X receptor antagonist and/or at least one Vitamin D receptor antagonist, preferably, for a time period of 0.1-50%, preferably, 0.1-25%, more preferably, 0.1-15%, of an entire ex-vivoculturing period of the hematopoietic mononuclear cells.

According to still further features in the described preferred embodiments, the retinoic acid receptor antagonist is selected from the group consisting of: AGN 194310; AGN 193109; 3-4-Methoxy-phenylsulfanyl)-3-methyl-butyric acid; 6-Methoxy-2,2-dimethyl-thiochroman-4-one, 2,2-Dimethyl-4-oxo-thiochroman-6-15 yltrifluoromethane-sulfonate; Ethyl 4-((2,2 dimethyl-4-oxo-thiochroman-6-yl)ethynyl)-benzoate; Ethyl 4-((2,2-dimethy 1-4-triflouromethanensulfonyloxy-(2H)-thiochromen-6-yl)ethynyl)-benzoate(41); Thiochromen-6-yl]-ethynyl]-benzoate(yl); (p-[(E)-2-[3′,4′-Dihydro-4,4′-dimethyl-7′-(heptyloxy)-2′H-1-benzothiopyran-6yl] propenyl] benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-octa-2,4,6-trienoic acid; 2E,4E,6E-[7-(3,5-Di-t-butyl-4-n-pentoxyphenyl)-3-methyl]-octa-2,4,6-trienoic acid; 2E,4E,6E-[7-(3,5-Di-t-butyl-4-n-hexoxyphenyl)-3-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-[3-t-butyl-5-(1-phenyl-vinyl)-phenyl]-3-methyl-octa-2,4,6-trienoic acid; 2E,4E,6E-[7-(3,5-Di-t-butyl-4-{[4,5-.sup.3H.sub.2]-n-pentoxy} phenyl)-3-methyl]-octa-2,4,6-trienoic acid; (2E,4E)-(1RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl]-3-methyl-penta-2,4-dienoic acid; (2E,4E)-(1RS,2RS)-5-[2-(3,5-di-tert.butyl-2-butoxy-phenyl)-cyclopropyl]-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido) benzoic acid; (2E,4E)-3-methyl-5-[(1 S,2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′H-1-benzothiopyran-6′-yl]propenyl]benzoic acid; 1′, 1′-dioxide, 4-(7,7,10,10-Tetramethyl-1-pyridin-3-ylmethyl-4,5,7,8,9, 10-hexahydro-1H-naphto[2,3-g]indol-3-yl)-benzoic acid; (2E,4E,6Z)-7-[3,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid (2E,4E,6Z)-7-(3-n-propoxy-5,6,7,8-tetrahydro-5,5,8,8-tetramethylnaphthalene-2-yl)-3-methylocta-2,4,6-trienoic acid, 4-(5H-2,3(2,5 dimethyl-2,5-hexano)-5-n-propyldibenzo[b,e][1,4]diazepin-11-yl)benzoic acid, 4-(5H-2,3-(2,5-dimethyl-2, 5-hexano)-5methyl-8-nitrodibenzo[b,e][1,4]diazepin-11-yl)benzoic acid, 4-{[4-(4-Ethylphenyl)2,2-dimethyl-(2H)-thiochromen-6-yl]ethynyl}benzoic acid, 4-[4-2methyl-1,2-dicarba-closo-dodecaboran-1-yl-phenylcarbamoyl]benzoic acid, 4-[4,5,7,8,9,10-hexahydro-7,7,10,10-tetramethyl-1-(3-pyridylmethyl)-anthra[1,2-b]pyrrol-3-yl]benzoic acid, (3-pyridylmethyl)-]5-thiaanthra[2,1-b]pyrrol-3-yl)benzoic acid, and (3-pyridylmethyl)-anthra[2m 1-d]pyrazol-3-yl]benzoic acid.

According to still further features in the described preferred embodiments, the retinoid X receptor antagonist is selected from the group consisting of: LGN 100572, LGN 100574, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3 [-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl] benzoic acid, 4-[1-(3,5, 5,8,8-pentamethyl-5,6,7,8-tetrahydro-2-naphthyl)ethenyl] benzoic acid, 4-[1(3,5,5,8,8-pentamethyl-5,6,7,8-tetrahydro-2-naphthyl)cyclopropyl] benzoic acid, 4-[1-(3,5,5,8,8-pentamethyl-5,6,7,8-tetrahydro-2-naphthyl)ethenyl] benzenete trazole, 2-[1-(5,5,8,8-tetramethyl-5,6,7,8-tetrahydro-2-naphthyl) ethenyl]pyridine-5-carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8,8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl) cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5, 5,8,8-pentamethyl-5,6,7,8-tetrahydro-2-naphthyl)ethenyl]-N-(4-hydroxyphenyl) benzamide, 2-[1-(3,5,5,8,8-Pentamethyl-5,6,7,8-tetrahydro-2-naphthyl) ethenyl] pyridine-5-carboxylic acid, 2,4,6-(3,5,5,8,8-Pentamethyl-5, 6,7,8-tetrahydro-2-naphthyl)cyclopropyl]pyridine-5-carboxylic acid, 4-[(3,5, 5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl) carbonyl]benzoic acid propyloxime, 4-[(3,5,5,8,8-pentamethyl-5,6,7,8-terrahydro-2-naphthyl)carbonyl]benzoic acid cyanoimine,154-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e] [1,4]diazepin-11-yl)benzoic acid.

According to still further features in the described preferred embodiments, the Vitamin D receptor antagonist is selected from the group consisting of: 1 alpha, 5-(OH)-D3-26,23 lactone; 1 alpha, 25-dihydroxyvitamin D (3); the 25-carboxylic ester ZK159222; (23S)-25-dehydro-1 alpha-OH-D (3); (23R)-25-dehydro-1 alpha-OH-D (3); 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-30 secocholesta-5,7,10(19),22-tetraene-25-carboxylate).

According to still further features in the described preferred embodiments, the PI 3-kinase inhibitor is selected from the group consisting of wortmannin and LY294002.

The copper chelate(s) or chelator(s) used in the various aspects of the present invention described hereinabove preferably comprise a polyamine chelator.

According to further features in preferred embodiments of the invention described below, the polyamine chelator is capable of forming an organometallic complex with a transition metal other than copper. The transition metal can be, for example, zinc, cobalt, nickel, iron, palladium, platinum, rhodium and ruthenium.

According to still further features in the described preferred embodiment the polyamine chelator is a linear polyamine. Preferably, the linear polyamine has a general formula I:
HX-Am-(Y₁B₁)₁ . . . (YnBn)ₙ-ZH Formula 1
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; and B₁ and Bn are each independently an alkylene chain having between 1 and 20 substituted and/or non-substituted carbon atoms, provided that at least one of the X, Z, Y₁ and Yn is a —NH group and/or at least one of the carbon atoms in the alkylene chains is substituted by an amine group.

According to still further features in the described preferred embodiments, A is an alkylene chain having a general formula II: 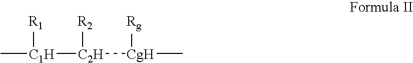
wherein g is an integer that equals 0 or 3-10; and each of R₁, R₂ and Rg is independently selected from the group consisting of hydrogen, alkyl, alkenyl, alkynyl, aryl, cycloalkyl, heteroalicyclic, heteroaryl, halo, amino, alkylamino, arylamino, cycloalkylamino, heteroalicyclic amino, heteroarylamino, hydroxy, alkoxy, aryloxy, azo, C-amido, N-amido, ammonium, thiohydroxy, thioalkoxy, thioaryloxy, sulfonyl, sulfinyl, N-sulfonamide, S-sulfonamide, phosphonyl, phosphinyl, phosphonium, carbonyl, thiocarbonyl, C-carboxy, O-carboxy, C-thiocarboxy, O-thiocarboxy, N-carbamate, O-carbamate, N-thiocarbamate, O-thiocarbamate, urea, thiourea, botate, borane, boroaza, silyl, siloxy, silaza, aquo, alcohol, peroxo, amine oxide, hydrazine, alkyl hydrazine, aryl hydrazine, nitric oxide, cyanate, thiocyanate, isocyanate, isothiocyanate, cyano, alkylnitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl saulfinic acid, alkyl thiol carboxylic acid, aryl thiol carboxylic acid, alkyl thiol thiocarboxylic acid, aryl thiol thiocarboxylic acid, carboxylic acid, alkyl carboxylic acid, aryl carboxylic acid, sulfate, sulfite, bisulfite, thiosulfate, thiosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphone sulfide, alkyl phosphonic acid, aryl phosphonic acid, alkyl phosphinic acid, aryl phosphinic acid, phosphate, thiophosphate, phosphite, pyrophosphite, triphosphate, hydrogen phosphate, dihydrogen phosphate, guanidino, S-dithiocarbamate, N-dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraarylborate, tetraalkyl borate, tartarate, salicylate, succinate, citrate, ascorbate, saccharirate, amino acid, hydroxamic acid and thiotosylate.

According to still further features in the described preferred embodiments, each of B1 and Bn is independently an alkylene chain having a general formula III 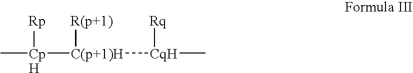
wherein p is an integer that equals 0 or g+1; q is an integer from g+2 to g+20; and each of Rp, Rp+1 and Rq is independently selected from the group consisting of the substituents described hereinabove with respect to R₁, R₂ and Rg.

According to still further features in the described preferred embodiments at least one of C₁, C₂ and Cg and/or at least one of Cp, Cp+1 and Cq is a chiral carbon atom.

A preferred linear polyamine according to the present invention is tetraethylenepentamine.

According to still further features in the described preferred embodiments the polyamine chelator is a cyclic polyamine, such as cyclam.

According to still further features in the described preferred embodiments the cyclic polyamine has a general formula IV: 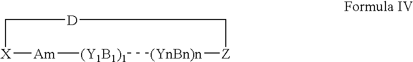
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; B₁ and Bn are each independently an alkylene chain having between 1 and 20 substituted and/or non-substituted carbon atoms; and D is a bridging group having a general formula V:
U-W-V Formula V
whereas U and V are each independently selected from the group consisting of substituted hydrocarbon chain and non-substituted hydrocarbon chain; and W is selected from the group consisting of amide, ether, ester, disulfide, thioether, thioester, imine and alkene,
provided that at least one of the X, Z, Y₁ and Yn is a —NH group and/or at least one of the carbon atoms in the alkylene chains is substituted by an amine group.

According to still further features in the described preferred embodiments, A and each of B1 and Bn in Formula IV are alkylene chains having the general formulas II and III, as is described hereinabove.

According to still further features in the described preferred embodiments the cyclic polyamine has a general formula selected from the group consisting of: 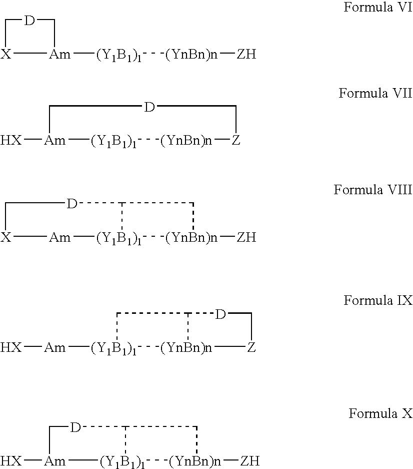
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; B1 and Bn are each independently an alkylene chain having between 1 and 20 substituted and/or non-substituted carbon atoms; and D is a bridging group having a general formula V, as described hereinabove, and further wherein should the D is attached at one end to A (Formulas VI, VII and X), the U or the V are being attached to one carbon atom in the alkylene chain and should the D is attached at one end to B1 or Bn (Formulas VIII, IX and X), the U or the V are being attached to one carbon atom in the alkylene chain, provided that at least one of the X, Z, Y₁ and Yn is a —NH group and/or at least one of the carbon atoms in the alkylene chains is substituted by an amine group.

The alkylene chains A, B1 and Bn are preferably as described hereinabove.

According to still further features in the described preferred embodiments the polyamine chelator includes at least one linear polyamine and at least one cyclic polyamine.

Such a polyamine chelator preferably has a general formula XI:
{(E1)_{f}[Q₁-(G₁)_{g}]}ₕ-{(E₂)ᵢ-[Q₂-(G₂)ⱼ]}ₖ-. . . . -{(Eₙ)₁-[Qₙ-(Gₙ)ₒ]}ₜ Formula XI
wherein n is an integer greater than 1; each of f, g, h, i, j, k, l, o and t is independently an integer from 0 to 10; each of E₁, E₂ and En is independently a linear polyamime as is described hereinabove; each of G₁, G₂ and Gn is independently a cyclic polyamine as is described hereinabove; and each of Q₁, Q₂ and Qn is independently a linker linking between two of the polyamines,
provided that at least one of the Q₁, Q₂ and Qn is an amine group and/or at least one of the linear polyamine and the cyclic polyamine is having at least one free amine group.

According to still further features in the described preferred embodiments each of Q₁, Q₂ and Qn is independently selected from the group consisting alkylene, alkenylene, alkynylene, arylene, cycloalkylene, hetroarylene, amine, azo, amide, sulfonyl, sulfinyl, sulfonamide, phosphonyl, phosphinyl, phosphonium, ketoester, carbonyl, thiocarbonyl, ester, ether, thioether, carbamate, thiocarbamate, urea, thiourea, borate, borane, boroaza, silyl, siloxy and silaza.

According to still further features in the described preferred embodiments the polyamine chelator is selected from the group consisting of ethylendiamine, diethylenetriamine, triethylenetetramine, triethylenediamine, tetraethylenepentamine, aminoethylethanolamine, aminoethylpiperazine, pentaethylenehexamine, captopril, penicilamine, N,N′-bis(3-aminopropyl)-1,3-propanediamine, N,N′-Bis-(2-animoethyl)-1,3-propanediamine, 1,7-dioxa-4,10-diazacyclododecane, 1,4,8,11-tetraaza cyclotetradecane-5,7-dione, 1,4,7-triazacyclononane, 1-oxa-4,7,10-triazacyclododecane, 1,4,8,12-tetraazacyclopentadecane, and 1,4,7,10-tetraazacyclododecane.

The present invention successfully addresses the shortcomings of the presently known configurations by providing methods of expanding hematopoietic stem cells without first enriching hematopoietic mononuclear cells for stem cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b illustrates the effect of TEPA chelator on the expansion of CD34⁺ hematopoietic stem cells in a culture of hematopoietic mononuclear cells. Cord-blood mononuclear cells (MNCs) were seeded in culture-bags in the presence of cytokines, and were either supplemented with TEPA chelator (MNC-TEPA), or not supplemented with TEPA chelator (MNC control). For comparison, purified CD34⁺ cells were similarly seeded in culture-bags in the presence of cytokines with no supplementation of TEPA chelator (CD34⁺ culture). All cultures were incubated for 12 weeks and at weekly intervals, the CD34⁺ cells were purified from cultures using miniMacs columns and enumerated; 10 FIG. 2 illustrates the FACS-analysis of the density of CD34⁺CD38⁻ cells in the untreated NMCs, TEPA-treated MNCs and CD34⁺ cell cultures described above; and

FIG. 3 presents the comparative numbers of colony-forming cells (CFUs) measured from the untreated MNCs, TEPA-treated MNCs and CD34⁺ cell cultures described above, at weekly intervals.

### DESCRIPTION OF THE PREFERRED EMBODIMENTS

The present invention is of methods of ex-vivo expanding a population of hematopoietic stem cells present, as a minor fraction, in hematopoietic mononuclear cells, without first enriching the stem cells, while at the same time, substantially inhibiting differentiation of the hematopoietic stem cells. The present invention can be used to efficiently provide ex-vivo expanded populations of hematopoietic stem cells, using hematopoietic mononuclear cells that comprise a major fraction of hematopoietic committed cells and a minor fraction of the hematopoietic stem and progenitor cells as a source of stem cells, without prior enrichment of the hematopoietic mononuclear cells for stem cells. The expanded populations of hematopoietic stem cells of the present invention can be used in, for example, hematopoietic cell transplantation, in generation of stem cells suitable for genetic manipulations for cellular gene therapy, as well as in additional application such as, but not limited to, adoptive immunotherapy, implantation of stem cells in an in vivo cis-differentiation and trans-differentiation settings, as well as, ex-vivo tissue engineering in cis-differentiation and trans-differentiation settings.

The methods of the present invention utilize various molecules (also referred to herein as agents), that interfere with CD38 expression and/or activity and/or with intracellular copper content, for inducing the ex-vivo expansion of hematopoietic stem cell populations described above, thereby providing an efficient, simplified and yet versatile technology for ex-vivo expansion of hematopoietic stem cells.

The principles and operation of the present invention may be better understood with reference to the drawings and accompanying descriptions and examples.

Before explaining at least one embodiment of the invention in detail, it is to be understood that the invention is not limited in its application to the details of construction and the arrangement of the components set forth in the following description or illustrated in the Examples section. The invention is capable of other embodiments or of being practiced or carried out in various ways. Also, it is to be understood that the phraseology and terminology employed herein is for the purpose of description and should not be regarded as limiting.

As is discussed hereinabove, WO 99/40783, WO 00/18885 and Peled etsal, Brit. J. Haematol. 116:655 2002 teach that cellular copper is involved in modulating the balance between self-renewal and differentiation of hematopoietic progenitor cells. According to the teachings of these references, the addition of transition metal chelators that are capable of binding copper, such as, for example, the linear polyamine tetraethylenepentamine, to CD34+ cell cultures in the presence of early acting cytokines reduced cell copper content by 30% and extended the duration of the long-term cultures in terms of long-term CFU and CD34+ cell expansion. These references hence teach methods of expanding stem cell populations, ex-vivo in the presence of transition metal chelators, copper chelators in particular, and further teach the use of the obtained expanded stem cell populations in various applications.

PCT/IL03/00062 discloses that copper chelates, namely, copper chelators that are complexed with a copper ion, also promote proliferation and inhibit differentiation of stem and progenitor cells when added to the culture media of such cells. According to the teachings of PCT/IL03/00062, these finding suggest that this effect of copper chelates on proliferation and differentiation of stem and progenitor cells is not associated solely with the content of cellular copper but rather with additional regulatory pathways.

PCT/IL03/00064 and U.S. Provisional Patent Application No. 60/452,545, which are incorporated by reference as if fully set forth herein, disclose that a series of molecules that are capable of interfering with CD38 expression and/or activity, repress the process of differentiation of stem cells and stimulates and prolongs, for up to 16-18 weeks, the phase of active cell proliferation and expansion (renewal) ex-vivo, in a reversible manner. Hence, these references teach methods of expanding stem cell populations ex-vivo, which involve the addition of agents that either down-regulate CD38 expression or inhibit the activity of CD38 to the culture media of stem cells. The methods disclosed in PCT/IL03/00064 and U.S. Provisional Patent Application No. 60/452,545, therefore utilize molecules such as retinoic acid receptor antagonists of the RAR and RXR superfamilies, Vitamin D receptor antagonists, polynucleotides encoding antibodies such as anti CD38, anti retinoic acid receptor, anti retinoid X receptor, anti Vitamin D receptor, polynucleotides that are directed to cause degradation of endogenous polynucleotides encoding for these receptors, molecules that are capable of interfering with expression and/or activity of PI 3-kinase land CD38 inhibitors such as nicotinamide and its related compounds.

Hence, WO 99/40783, WO 00/18885, PCT/IL03/00064 and U.S. Provisional Patent Application No. 60/452,545 all teach the use of various molecules that modulate, via diverse pathways and/or mechanisms, the balance between self-renewal and differentiation of stem cells, hematopoietic stem cells in particular, in method for ex-vivo expanding of stem cell populations. However, unless otherwise indicated, the regulation of self-renewal and differentiation of stem cells by these molecules is obtained, according to the teachings of these references, when the cultured cells are first enriched for stem and/or progenitor cells and hence, in line with other present day technologies in this field, require preliminary stem cells enrichment.

While reducing the present invention to practice, it was surprisingly and unexpectedly found that molecules such as copper chelators, copper chelates and retinoic acid receptor (RAR) antagonists repress differentiation and stimulate and prolong proliferation of hematopoietic stem cells when the source of cells includes the entire fraction of mononuclear blood cells, namely non-enriched stem cells.

As is described in the Background section hereinabove, although being highly advantageous, presently there is no disclosed technology by which to expand non-enriched stem cells. Therefore, the technology presented and exemplified herein, involving methods of ex-vivo expanding hematopoietic stem cell populations devoid of prior stem cells enrichment, provides for efficient, simplified and cost-effective methods of obtaining ex-vivo expanded hematopoietic stem cell populations. The expanded hematopoietic stem cell populations obtained by the technology presented herein can be used in various application, the following lists a few:

Hematopoietic cell transplantation: Transplantation of hematopoietic cells has become the treatment of choice for a variety of inherited or malignant diseases. While early transplantation procedures utilized the entire bone marrow (BM) population, recently, more defined populations, enriched for stem cells (CD34⁺ cells) have been used (Van Epps D E, et al. Harvesting, characterization, and culture of CD34+ cells from human bone marrow, peripheral blood, and cord blood. Blood Cells 20:411, 1994). In addition to the marrow, such cells could be derived from other sources such as peripheral blood (PB) and neonatal umbilical cord blood (CB) (Emerson S G. Ex-vivo expansion of hematopoietic precursors, progenitors, and stem cells: The next generation of cellular therapeutics. Blood 87:3082, 1996). Compared to BM, transplantation with PB cells shortens the period of pancytopenia and reduces the risks of infection and bleeding (Brugger W, et al. Reconstitution of hematopoiesis after high-dose chematotherapy by autologous progenitor cells generated in-vivo. N Engl J Med 333:283, 1995; Williams S F, et al. Selection and expansion of peripheral blood CD34+ cells in autologous stem cell transplantation for breast cancer. Blood 87: 87, 1996; Zimmerman R M, et al. Large-scale selection of CD34+ peripheral blood progenitors and expansion of neutrophil precursors for clinical applications. J Heamatotherapy 5:247, 1996).

An additional advantage of using PB for transplantation is its accessibility. The limiting factor for PB transplantation is the low number of circulating pluripotent stem/progenitor cells.

To obtain enough PB-derived stem cells for transplantation, these cells are “harvested” by repeated leukophoresis following their mobilization from the marrow into the circulation by treatment with chemotherapy and cytokines (Brugger W, et al. Reconstitution of hematopoiesis after high-dose chematotherapy by autologous progenitor cells generated in-vivo. N Engl J Med 333:283, 1995; Williams S F, et al. Selection and expansion of peripheral blood CD34+cells in autologous stem cell transplantation for breast cancer. Blood 87:1687, 1996). Such treatment is obviously not suitable for normal donors.

The use of ex-vivo expanded stem cells for transplantation has the following advantages (Koller M R, Emerson S G, Palsson B O. Large-scale expansion of human stem and progenitor cells from bone marrow mononuclear cells in continuous perfusion cultures. Blood 82:378, 1993; Lebkowski J S, et al. Rapid isolation and serum-free expansion of human CD34+ cells. Blood Cells 20:404, 1994):

It reduces the volume of blood required for reconstitution of an adult hematopoietic system and may obviate the need for mobilization and leukophoresis (Brugger W, et al. N Engl J Med 333:283, 1995).

It enables storage of small number of PB or CB stem cells for potential future use.

In the case of autologous transplantation of recipients with malignancies, contaminating tumor cells in autologous infusion often contribute to the recurrence of the disease (Brugger W, et al. N Engl J Med 333:283, 1995). Selecting land expanding CD34⁺ stem cells will reduce the load of tumor cells in the final transplant.

The cultures provide a significant depletion of T lymphocytes, which may be useful in the allogeneic transplant setting for reducing graft-versus-host disease.

Clinical studies indicate that transplantation of ex-vivo expanded cells derived from a small number of PB CD34⁺ cells can restore hematopoiesis in recipients treated with high doses of chemotherapy, although the results do not yet allow firm conclusions about long term in-vivo hematopoietic capabilities of these cultured cells (Brugger W, et al. N Engl J Med 333:283, 1995; Williams S F, et al. Blood 87:1687, 1996).

For successful transplantation, shortening of the duration of the cytopenic phase, as well as long-term engraftment, is crucial. Inclusion of intermediate and late progenitor cells in the transplant could accelerate the production of donor-derived mature cells thereby shortening the cytopenic phase. It is important, therefore, that ex-vivo expanded cells include, in addition to stem cells, more differentiated progenitor cells in order to optimize short-term recovery and long-term restoration of hematopoiesis. Expansion of intermediate and late progenitor cells, especially those committed to the neutrophilic and megakaryocytic lineages, concomitant with expansion of stem cells, should serve this purpose (Sandstrom C E, et al. Effects of CD34+ cell selection and perfusion on ex-vivo expansion of peripheral blood mononuclear cells. Blood 86:958, 1995).

Such cultures may be useful in restoring hematopoiesis in recipients with completely ablated bone marrow, as well as in providing a supportive measure for shortening recipient bone marrow recovery following conventional radio- or chemotherapies.

Prenatal diagnosis of genetic defects in scarce cells: Prenatal diagnosis involves the collection of embryonic cells from a pregnant woman, in utero, and analysis thereof for genetic defects. A preferred, non-invasive, means of collecting embryonic cells involves separation of embryonic nucleated red blood cell precursors that have infiltrated into peripheral maternal circulation. However, since the quantities of these cells are quite scarce, a further application of the present invention would be the expansion of such cells according to methods described herein, prior to analysis. The present invention, therefore, offers a means to expand embryonic cells for applications in prenatal diagnosis.

Gene therapy: For successful long-term gene therapy, a high frequency of genetically modified stem cells with transgenes stably integrated within their genome, is an obligatory requirement. In BM tissue, while the majority of cells are cycling progenitors and precursors, stem cells constitute only a small fraction of the cell population and most of them are in a quiescent, non-cycling state. Viral-based (e.g., retroviral) vectors require active cell division for integration of the transgene into the host genome. Therefore, gene transfer into fresh BM stem cells is highly inefficient. The ability to expand a purified population of stem cells and to regulate their cell division ex-vivo would provide for an increased probability of their genetic modification (Palmiter R D. Regulation of metallothionein genes by heavy metals appears to be mediated by a zinc-sensitive inhibitor that interacts with a constitutively active transcription factor, MTF-1. Proc Natl Acad Sci USA 91(4): 1219-1223, 1994).

Adoptive immunotherapy: Ex-vivo-expanded, defined lymphoid subpopulations have been studied and used for adoptive immunotherapy of various malignancies, immunodeficiencies, viral and genetic diseases (Freedman A R, et al. Generation of T lymphocytes from bone marrow CD34+ cells in-vitro. Nature Medicine 2: 46, 1996; Heslop H E, et al. Long term restoration of immunity against Epstein-Barr virus infection by adoptive transfer of gene-modified virus-specific T lymphocytes. Nature Medicine 2: 551, 1996; Protti M P, et al. Particulate naturally processed peptides prime a cytotoxic response against human melanoma in-vitro. Cancer Res 56: 1210, 1996).

The treatment enhances the required immune response or replaces deficient functions. This approach was pioneered clinically by Rosenberg et al. (Rosenberg S A, et al. Prospective randomized trial of high-dose interleukin-2 alone or in conjunction with lymphokine-activated killer cells for the treatment of patients with advanced cancer. J Natl Cancer Inst 85: 622, 1993) using a large number of autologous ex-vivo expanded non-specific killer T cells, and subsequently ex-vivo expanded specific tumor infiltrating lymphocytes.

Functionally active, antigen-presenting cells could be grown from a starting population of CD34⁺ PB cells in cytokine-supported cultures, as well. These cells can present soluble protein antigens to autologous T cells in-vitro and, thus, offer new prospects for the immunotherapy of minimal residual disease after high dose chemotherapy. Ex-vivo expansion of antigen-presenting dendritic cells has been studied as well, and is an additional promising application of the currently proposed technology (Bernhard H, et al. Generation of immunostimulatory dendritic cells from human CD34+ hematopoietic progenitor cells of the bone marrow and peripheral blood. Cancer Res 10: 99, 1995; Fisch P, et al. Generation of antigen-presenting cells for soluble protein antigens ex-vivo from peripheral blood CD34+ hematopoietic progenitor cells in cancer patients. Eur J Immunol 26: 595, 1996; Siena S, et al. Massive ex-vivo generation of functional dendritic cells from mobilized CD34+ blood progenitors for anticancer therapy. Expt Hematol 23:1463, 1996).

As is discussed in brief hereinabove and is further detailed in WO 99/40783, WO 00/18885, PCT/IL03/00064 and U.S. Provisional Patent Application No. 60/452,545, copper chelators, copper chelates and retinoid receptor antagonists, each modulate the self-renewal of stem cells via a different pathway, effecting different cellular events that lead to reduced differentiation and extended proliferation of stem cells. These molecules therefore represent a wide variety of molecules that are capable of inducing the effect of expanding a hematopoietic stem cells population that is present in a mixed hematopoietic cells population.

Hence, according to one aspect of the present invention there is provided a method of ex-vivo expanding a population of hematopoietic stem cells, while at the same time, substantially inhibiting differentiation of the stem cells ex-vivo. The method according to this aspect of the present invention is effected by providing hematopoietic mononuclear cells which comprise a major fraction of hematopoietic committed cells and a minor fraction of hematopoietic stem and progenitor cells, with ex-vivo culture conditions for ex-vivo cell proliferation and, at the same time, for reducing an expression and/or activity of CD38, thereby expanding a population of hematopoietic stem cells, while at the same time, substantially inhibiting differentiation of the hematopoietic stem cells ex-vivo.

As used herein, the phrase “hematopoietic mononuclear cells” refers to the entire repetoir of white blood cells present in a blood sample. In a healthy human being, the white blood cells comprise a mixture of hematopoietic lineages committed and differentiated cells (typically over 99% of the mononuclear cells are lineages committed cells) including, for example: Lineage committed progenitor cells CD34⁺CD33⁺ (myeloid committed cells), CD34⁺CD3⁺ (lymphoid committed cells) CD34⁺CD41⁺ (megakaryocytic committed cells) and differentiated cells—CD34⁻CD33⁺ (myeloids, such as granulocytes and monocytes), CD34⁻CD3⁺, CD34⁻CD19⁺ (T and B cells, respectively), CD34⁻CD41⁺ (megakaryocytes), and hematopoietic stem and early progenitor cells such as CD34⁺Lineage negative (Lin⁻), CD34-Lineage negative CD34+CD38⁻ (typically less than 1%).

The phrase “hematopoietic mononuclear cells which comprise a major fraction of hematopoietic committed cells and a minor fraction of hematopoietic stem and progenitor cells” is used herein to describe any portion of the white blood cells fraction, in which the majority of the cells are hematopoietic committed cells, while the minority of the cells are hematopoietic stem and progenitor cells, as these terms are further defined hereinunder.

Hematopoietic mononuclear cells are typically obtained from a blood sample by applying the blood sample onto a Ficoll-Hypaque layer and collecting, following density-cussion centrifugation, the interface layer present between the Ficoll-Hypaque and the blood serum, which interface layer essentially entirely consists of the white blood cells present in the blood sample.

As used herein, the phrase “hematopoietic committed cells” refers to differentiated hematopoietic cells that are committed to a certain hematopoietic cell lineage and hence can develop under physiological conditions substantially only to this specific hematopoietic lineage.

As used herein, the phrase “hematopoietic stem cells” refers to pluripotent hematopoietic cells that, given the right growth conditions, may develop to any cell lineage present blood. This phrase, as used herein, refers both to the earliest renewable hematopoietic cell populations responsible for generating cell mass in the blood (e.g., CD34⁻/AC133⁺, CD34⁻/AC133⁻/Lineage⁻, CD34⁺/AC133⁺cells) and the very early hematopoietic progenitor cells, which are somewhat more differentiated, yet are not committed and can readily revert to become a part of the earliest renewable hematopoietic cell population (e.g., CD34⁺ cells, especially CD34⁺CD38⁻ -cells).

In normal human, most of the hematopoietic pluripotent stem cells and the lineage committed progenitor cells are CD34⁺. The majority of cells are CD34⁺CD38⁺, with aminority of cells (<10%) being CD34⁺CD38⁻.

The CD34⁺CD38⁻ stem cells fraction identifies the most immature hematopoietic cells, which are capable of self-renewal and multilineage differentiation. This fraction contains more long-term culture initiating cells (LTC-IC) pre-CFU and exhibits longer maintenance of the stem cells and delayed proliferative response to cytokines as compared with the CD34⁺CD38⁺ cell fraction.

Presently, hematopoietic stem cells are obtained by further enrichment of the hematopoietic mononuclear cells obtained by differential density centrifugation as described above. This further enrichment process is typically performed by immuno-separation such as immunomagnetic-separation or FACS and results in a cell fraction that is enriched for hematopoietic stem cells.

Hence, using hematopoietic mononuclear cells as a direct source for obtaining expanded population of hematopoietic stem cells circumvents the need for stem cell enrichment prior to expansion, thereby substantially simplifying the process in terms of both efficiency and cost.

As used herein the term “inhibiting” refers to slowing, decreasing, delaying, preventing or abolishing.

As used herein the term “differentiation” refers to relatively generalized or specialized changes during development. Cell differentiation of various lineages is a well-documented process and requires no further description herein. As used herein the term differentiation is distinct from maturation which is a process, although some times associated with cell division, in which a specific cell type mature to function and then dies, e.g., via programmed cell death.

The phrase “cell expansion” is used herein to describe a process of cell proliferation substantially devoid of cell differentiation. Cells that undergo expansion hence maintain their cell renewal properties and are oftentimes referred to herein as renewable cells, e.g., renewable stem cells.

Expansion of hematopoietic stem cells using hematopoietic mononuclear cells as a source for the hematopoietic stem cells, as taught by the present invention, therefore result in converting the minor fraction (of less than 1%) of hematopoietic stem and progenitor cells present in the mononuclear cells into at least the major, if not the sole hematopoietic cells population post expansion, whereby in the course of stem cells expansion the committed cells are either substantially diluted and/or die.

As used herein the term “ex-vivo” refers to a process in which cells are removed from a living organism and are propagated outside the organism (e.g., in a test tube). As used herein, the term “ex-vivo”, however, does not refer to a process by which cells known to propagate only in-vitro, such as various cell lines (e.g., HI-60, MEL, HeLa, etc.) are cultured. In other words, cells expanded ex-vivo according to the present invention do not transform into cell lines in that they eventually undergo differentiation.

Providing the ex-vivo grown cells with conditions for ex-vivo cell proliferation include providing the cells with nutrients and preferably with one or more cytoknes, as is further detailed hereinunder.

As mentioned hereinabove, concomitant with treating the hematopoietic mononuclear cells with conditions which allow the cells to proliferate ex-vivo, the cells are short-term treated or long-term treated to reduce the expression and/or activity of CD38.

In one embodiment of the present invention, reducing the activity of CD38 is effected by providing the cells with an agent that inhibits CD38 activity (i.e., a CD38 inhibitor).

As used herein a “CD38 inhibitor” refers to an agent which is capable of downregulating or suppressing CD38 activity in stem cells.

A CD38 inhibitor according to this aspect of the present invention can be a “direct inhibitor” which inhibits CD38 intrinsic activity or an “indirect inhibitor” which inhibits the activity or expression of CD38 signaling components (e.g., the cADPR and ryanodine signaling pathways) or other signaling pathways which are effected by CD38 activity.

According to presently known embodiments of this aspect of the present invention, nicotinamide is a possible CD38 inhibitor.

Hence, in one embodiment, the method according to this aspect of the present invention is effected by providing the hematopoietic mononuclear cells either with nicotinamide itself, or with a nicotinamide analog, a nicotinamide or a nicotinamide analog derivative or a nicotinamide or a nicotinamide analog metabolite.

As used herein, the phrase “nicotinamide analog” refers to any molecule that is known to act similarly to nicotinamide. Representative examples of nicotinamide analogs include, without limitation, benzamide, nicotinethioamide (the thiol analog of nicotinamide), nicotinic acid and α-amino-3-indolepropionic acid.

The phrase “a nicotinamide or a nicotinamide analog derivative” refers to any structural derivative of nicotinamide itself or of an analog of nicotinamide. Examples of such derivatives include, without limitation, substituted benzamides, substituted nicotinamides and nicotinethioamides and N-substituted nicotinamides and nicotinthioamides.

The phrase “a nicotinamide or a nicotinamide analog metabolite” refers to products that are derived from nicotinamide or from analogs thereof such as, for example, NAD, NADH and NADPH.

Alternatively, a CD38 inhibitor according to this aspect of the present invention can be an activity-neutralizing antibody that binds, for example, to the CD38 catalytic domain, thereby inhibiting CD38 catalytic activity. It will be appreciated, though, that since CD38 is an intracellular protein measures are taken to use inhibitors which may be delivered through the plasma membrane. In this respect a fragmented antibody such as a Fab fragment (described hereinunder) is preferably used.

The term “antibody” as used in this invention includes intact molecules as well as functional fragments thereof, such as Fab, F(ab′)₂, and Fv that are capable of binding to macrophages. These functional antibody fragments are defined as follows:

Fab, the fragment which contains a monovalent antigen-binding fragment of an antibody molecule, can be produced by digestion of whole antibody with the enzyme papain to yield an intact light chain and a portion of one heavy chain;
Fab′, the fragment of an antibody molecule that can be obtained by treating whole antibody with pepsin, followed by reduction, to yield an intact light chain and a portion of the heavy chain; two Fab′ fragments are obtained per antibody molecule;
(Fab′)₂, the fragment of the antibody that can be obtained by treating whole antibody with the enzyme pepsin without subsequent reduction; F(ab′)₂ is a dimer of two Fab′ fragments held together by two disulfide bonds;
Fv, defined as a genetically engineered fragment containing the variable region of the light chain and the variable region of the heavy chain expressed as two chains; and
Single chain antibody (“SCA”), a genetically engineered molecule containing the variable region of the light chain and the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York, 1988, incorporated herein by reference).

Antibody fragments according to the present invention can be prepared by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio[Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins recipient antibody in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source that is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,42; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Alternatively, the method according to this aspect of the present invention can be effected by providing the ex-vivo cultured hematopoietic mononuclear cells with an agent that downregulates CD38 expression.

An agent that downregulates CD38 expression refers to any agent which affects CD38 synthesis (decelerates) or degradation (accelerates) either at the level of the mRNA or at the level of the protein. For example, a small interfering polynucleotide molecule which is designed to down regulate the expression of CD38 can be used according to this aspect of the present invention.

An example for a small interfering polynucleotide molecule which can down-regulate the expression of CD38 is a small interfering RNA or siRNA, such as for example, the morpholino antisense oligonucleotides described by in Munshi et al. (Munshi C B, Graeff R, Lee H C, J Biol Chem 2002 Dec. 20;277(51):49453-8), which includes duplex oligonucleotides which direct sequence specific degradation of mRNA through the previously described mechanism of RNA interference (RNAi) (Hutvagner and Zamore (2002) Curr. Opin. Genetics and Development 12:225-232).

As used herein, the phrase “duplex oligonucleotide” refers to an oligonucleotide structure or mimetics thereof, which is formed by either a single self-complementary nucleic acid strand or by at least two complementary nucleic acid strands. The “duplex oligonucleotide” of the present invention can be composed of double-stranded RNA (dsRNA), a DNA-RNA hybrid, single-stranded RNA (ssRNA), isolated RNA (i.e., partially purified RNA, essentially pure RNA), synthetic RNA and recombinantly produced RNA.

Preferably, the specific small interfering duplex oligonucleotide of the present invention is an oligoribonucleotide composed mainly of ribonucleic acids.

Instructions for generation of duplex oligonucleotides capable of mediating RNA interference are provided in www.ambion.com.

Hence, the small interfering polynucleotide molecule according to the present invention can be an RNAi molecule (RNA interference molecule).

Alternatively, a small interfering polynucleotide molecule can be an oligonucleotide such as a CD38-specific antisense molecule or a rybozyme molecule, further described hereinunder.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art.

Oligonucleotides used according to this embodiment of the present invention are those having a length selected from a range of 10 to about 200 bases preferably 15-150 bases, more preferably 20-100 bases, most preferably 20-50 bases.

The oligonucleotides of the present invention may comprise heterocyclic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos.: 4,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfonate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propyryl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

As described hereinabove, the oligonucleotides of the present invention are preferably antisense molecules, which are chimeric molecules. “Chimeric antisense molecules” are oligonucleotides, which contain two or more chemically distinct regions, each made up of at least one nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids. An example for such includes RNase H, which is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense molecules of the present invention may be formed as composite structures of two or more oligonucleotides, modified oligonucleotides, as described above. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein fully incorporated by reference.

The oligonucleotides of the present invention can further comprise a ribozyme sequence. Ribozymes are being increasingly used for the sequence-specific inhibition of gene expression by the cleavage of mRNAs. Several rybozyme sequences can be fused to the oligonucleotides of the present invention. These sequences include but are not limited ANGIOZYME specifically inhibiting formation of the VEGF-R (Vascular Endothelial Growth Factor receptor), a key component in the angiogenesis pathway, and HEPTAZYME, a rybozyme designed to selectively destroy Hepatitis C Virus (HCV) RNA, (Rybozyme Pharmaceuticals, Incorporated —WEB home page).

Further alternatively, a small interfering polynucleotide molecule, according to the present invention can be a DNAzyme.

DNAzymes are single-stranded catalytic nucleic acid molecules. A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002;4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Tier www.asgt.org). In another application, DNAzymes complementary to bcr-ab1 oncogenes were successful in inhibiting the oncogenes expression in leukemia cells, and lessening relapse rates in autologous bone marrow transplant in cases of CML and ALL.

Alternatively, as mentioned hereinabove and is further detailed in PCT/IL03/00064 and U.S. Provisional Patent Application No. 60/452,545, retinoid receptor superfamily inhibitors (e.g., antagonists, siRNA molecules, antisense molecules, antibodies, etc.) which downregulate or suppress retinoid receptor activity and/or expression can be used to downregulate CD38 expression.

Briefly, retinoid receptors such as retinoic acid receptor (RAR), retinoid X receptor (RXR) and vitamin D receptor (VDR) have been reported to be involved in the regulation of gene expression pathways associated with cell proliferation and differentiation and in particular in the regulation of CD38 expression [Kapil M., Teresa M., Taghi M., Michael A., Steven C., Maher A. Involvement of retinoic acid receptor mediated signaling pathway in induction of CD38 cell surface antigen, Blood. 1997; 89:3607-3614; Ueno H, Kizaki M, Matsushita H, Muto A, Yamato K, Nishihara T, Hida T, Yoshimura H, Koeffler H P, Ikeda Y. A novel retinoic acid receptor (RAR)-selective antagonist inhibits differentiation and apoptosis of HL-60 cells: implications of RAR alpha-mediated signals in myeloid leukemic cells. Leuk Res. 1998; 22:517-25]. Hence, preferred agents that downregulate CD38 expression according to the present invention include RAR antagonists, RXR antagonists and VDR antagonists or, alternatively, antagonists for reducing the capacity of the hematopoietic mononuclear cells in responding to retinoic acid, retinoid and/or Vitamin D.

As used herein the term “antagonist” refers to an agent that counteracts or abrogates the effects of an agonist or a natural ligand of a receptor. Further features relating to such antagonists are detailed hereinunder.

Further alternatively, as is described in detail in U.S. Provisional Patent Application No. 60/452,545, down regulation of CD38 expression can be obtained by downregulating the expression and/or activity of phosphatidyl inositol 3-kinase, which is also referred to herein throughout as PI 3-kinase. Briefly, it has been reported that PI 3-kinase plays a critical function in the activation of nuclear receptors such as the retinoid receptor superfamily and the vitamin D receptor, as an obligalory factor for proper receptor signaling pathways and is hence involved in cell differentiation.

Hence, agents that interfere with PI 3-kinase expression and/or activity are also preferred agents for downregulating CD38, according to the present invention. Representative examples of agents that inhibit PI 3-kinase activity include, but are not limited to, the known PI 3-kinase inhibitors wortmannin and LY294002, and analogs, derivatives, and metabolites thereof. Additional examples of PI 3-kinase inhibitors are described in U.S. Pat. No. 5,378,725, which is incorporated by reference as if fully set forth herein. Representative examples of agents that downregulate PI 3-kinase expression according to the present invention include, but are not limited to, polynucleotides, such as small interfering RNA molecules, antisense ribozymes and DNAzymes, as well as intracellular antibodies, using the methodologies described hereinabove with respect to downregulating CD38 expression.

Each of the agents described hereinabove may reduce the expression or activity of CD38 individually. However, the present invention aims to also encompass the use of any subcombination of these agents.

It will be appreciated that protein agents (e.g., antibodies) of the present invention can be expressed from a polynucleotide encoding same and provided to ex-vivo cultured hematopoietic mononuclear cells employing an appropriate gene delivery vehicle/method and a nucleic acid construct as is further described hereinunder.

Examples of suitable constructs include, but are not limited to pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

As the method of ex-vivo expanding a population of hematopoietic stem cells, according to this aspect of the present invention, is effected by modulating CD38 expression and/or activity, either at the protein level, using RAR, RXR or VDR antagonists, a PI-3 kinase inhibitor or a CD38 inhibitor such as nicotinamide and analogs thereof, or at the at the expression level via genetic engineering techniques, as is detailed hereinabove, there are further provided, according to the present invention, several preferred methods of ex-vivo expanding a population of hematopoietic stem cells of hematopoietic mononuclear cells.

In one particular, a method of ex-vivo expanding a population of hematopoietic stem cells, while at the same time, substantially inhibiting differentiation of the hematopoietic stem cells ex-vivo is effected by providing hematopoietic mononuclear cells which comprise a major fraction of hematopoietic committed cells and a minor fraction of hematopoietic stem and progenitor cells, with ex-vivo culture conditions for ex-vivo cell proliferation and, at the same time, for reducing a capacity of the hematopoietic mononuclear cells in responding to retinoic acid, retinoids and/or Vitamin D, so as to expand a population of hematopoietic stem cells, while at the same time, substantially inhibiting differentiation of the hematopoietic stem cells ex-vivo.

Reducing the capacity of the cells in responding to retinoic acid, retinoids and/or Vitamin D, or to retinoic acid, retinoid X and/or Vitamin D receptor signaling may be effected, for example, by the administration of chemical inhibitors, including receptor antagonists.

In another particular, the method of ex-vivo expanding a population of stem cells, while at the same time, substantially inhibiting differentiation of the stem cells ex-vivo is effected by providing hematopoietic mononuclear cells which comprise a major fraction of hematopoietic committed cells and a minor fraction of hematopoietic stem and progenitor cells, with ex-vivo culture conditions for ex-vivo cell proliferation and, at the same time, for reducing a capacity of the hematopoietic mononuclear cells in responding to signaling pathways involving the retinoic acid receptor, retinoid-X receptor and/or Vitamin D receptor, to thereby expand a population of hematopoietic stem cells, while at the same time, substantially inhibiting differentiation of the hematopoietic stem cells ex-vivo.

Reducing the capacity of the cells to respond to retinoic acid, retinoid X and/or Vitamin D receptor signaling events, includes treating the cells with antagonists supplied continuously or for a short-pulse period, and is effected by a diminution or abrogation of cellular signaling pathways through their respective, cognate receptors.

As is described and exemplified in PCT/IL01/00064, reducing the capacity of hematopoietic cells in responding to the disclosed signaling pathways is reversible, e.g., inherently reversible. In other words, cells expanded using the protocols of the present invention do not transform into cell lines. Hence, by exposing the cells following sufficient expansion to growth conditions by which differentiation is induced, one would be able to direct the ex-vivo differentiation of the cells to desired direction, including ex-vivo and in vivo cis- and trans-differentiation.

As used herein “cis-differentiation” refers to differentiation of adult stem cells into a tissue from which they were derived. For example, the differentiation of CD34+ hematopoietic cells to different committed/mature blood cells constitutes cis-differentiation.

As used herein “trans-differentiation” refers to differentiation of adult stem cells into a tissue from which they were not derived. For example, the differentiation of CD34+ hematopoietic cells to cells of different tissue origin, e.g., myocites constitutes trans-differentiation.

Reducing the capacity of the hematopoietic mononuclear cells in responding to the above antagonists and/or signaling pathways of the above receptors and kinase is effected by ex-vivo culturing hematopoietic mononuclear cells in a presence of an effective amount of at least one retinoic acid receptor antagonist, at least one retinoid X receptor antagonist and/or at least one Vitamin D receptor antagonist, preferably, for a time period of 0.1-50%, preferably, 0.1-25%, more preferably, 0.1-15%, of an entire ex-vivo culturing period of the hematopoietic mononuclear cells or for the entire period. In this respect it was surprisingly uncovered that an initial pulse exposure to an antagonist is sufficient to exert cell expansion long after the antagonist was removed from the culturing set up.

Final concentrations of the antagonists may be, depending on the specific application, in the micromolar or millimolar ranges. For example, within about 0.1 μM to about 100 mM, preferably within about 4 μM to about 50 mM, more preferably within about 5 μM to about 40 mM.

Many antagonists to RAR, RXR and VDR, which are usable in this and other aspects and embodiments of the present invention, are presently known.

Representative examples of such retinoic acid receptor antagonist include, without limitation, AGN 194310; AGN 109; 3-4-Methoxy-phenylsulfanyl)-3-methyl-butyric acid; 6-Methoxy-2,2-dimethyl-thiochroman4-one,2,2-Dimethyl-4-oxo-thiochroman-6-yltrifluoromethane-sulfonate; Ethyl 4-((2,2 dimethyl-4-oxo-thiochroman-6-yl)ethynyl)-benzoate; Ethyl 4-((2,2-dimethy 1-4-triflouromethanensulfonyloxy-(2H)-thiochromen-6-yl)ethynyl)-benzoate(41); Thiochromen-6-yl]-ethynyl]-benzoate(yl); (p-[(E)-2-[3′,4′-Dihydro-4,4′-dimethyl-7′-(heptyloxy)-2′H-1-benzothiopyran-6′yl] propenyl] benzoic acid 1′1′-dioxide; 2E,4E,6E-[7-(3,5-Di-t-butyl-4-n-butoxyphenyl)-3-methyl]-octa-2,4,6-trienoic acid; 2E,4E,6E-[7-(3,5-Di-t-butyl-4-n-propoxyphenyl)-3-methyl]-octa-2,4,6-trienoic acid; 2E,4E,6E-[7-(3,5-Di-t-butyl-4-n-pentoxyphenyl)-3-methyl]-octa-2,4,6-trienoic acid; 2E,4E,6E-[7-(3,5-Di-t-butyl-4-n-hexoxyphenyl)-3-methyl]-octa-2,4,6-trienoic acid; 2E,4E,6E-[7-(3,5-Di-t-butyl-4-n-heptoxyphenyl)-3-methyl]-octa-2,4,6-trienoic acid; 2E,4E,6E-[7-(3,5-Di-t-butyl-4-n-octoxyphenyl)-3-methyl]-octa-2,4,6-trienoic acid; (2E,4E,6E)-7-[3-t-butyl-5-(1-phenyl-vinyl)-phenyl]-3-methyl-octa-2,4,6-trienoic “d; 2E,4E,6E-[7-(3,5-Di-t-butyl-4-{[4,5-.sup.3H.sub.2]-n-pentoxy}phenyl)-3-methyl]-octa-2,4,6-trienoic acid; (2E,4E)-(1RS,2RS)-5-[2-(3,5-di-tert.butyl-2-ethoxy-phenyl)-cyclopropyl]-3-methyl-penta-2,4-dienoic acid ethyl ester; (2E,4E)-(1RS,2RS)-5-[2-(3,5-di-tert.butyl-2-ethoxy-phenyl)-cyclopropyl-3-methyl-penta-2,4-dienoic acid; (2E,4E)-(1RS,2RS)-5-[2-(3,5-di-tert.butyl-2-butoxy-phenyl)-cyclopropyl]-3-methyl-penta-2,4-dienoic acid; (2E,4E,6Z)-7-[3,5-di-tert.butyl-2-ethoxyphenyl]3-methyl-2,4,6-octatrienoic acid; (2E,4E,6Z)-7-[3,5-di-tert.butyl-2-butyloxyphenyl]-3-methyl-2,4,6-octatrienoic acid; 4-(5,6,7,8-tetrahydro-5,5,8,8-tetramethyl-2-naphthalene-carboxamido) benzoic acid; (2E,4E)-3-methyl-5-[(1S,2S)-2-(5,5,8,8-tetramethyl-5,6,7,8-tetrahydro-naphthalen-2-yl)-cyclopropyl]-penta-2,4-dienoic acid; p-[(E)-2-[3′,4′-Dihydro-4′,4′-dimethyl-7′-(heptyloxy)-2′H-1-benzothiopyran-6′-yl]propenyl]benzoic acid; 1′,1′-dioxide, 4-(7,7,10,10-Tetramethyl-1-pyridin-3-ylmethyl4,5,7,8,9,10-hexahydro-1 H-naphto[2,3-g]indol-3-yl)-benzoic acid; (2E,4E,6Z)-7-[3,5-di-tert.butyl-2-methoxyphenyl]-3-methyl-2,4,6-octatrienoic acid; (2E,4E,6Z)-7-[3,5-di-tert.butyl-2-ethoxyphenyl]-3-methyl-2,4,6-octatrienoic acid; (2E,4E,6Z)-7-[3,5-di-tert.butyl-2-hexyloxyphenyl]-3-methyl-2,4,6-octatrienoic acid; (2E,4E,6Z)-7-[3,5-di-tert.butyl-2-octyloxyphenyl]-3-methyl-2,4,6-octatrienoic acid; and (2E,4E)-(1RS,2RS)-5-[2-(3,5-di-tert-butyl-2-butoxy-phenyl)-cyclopropyl]-3-methyl-penta-2,4-dienoic acid (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5methyl-8-nitrodibenzo[b,e][1,4]diazepin-11-yl)benzoic acid, and 4-{[4-(4-Ethylphenyl)2,2-dimethyl-(2H)-thiochromen-6-yl]ethynyl}benzoic acid, and 4-[4-2methyl-1,2-dicarba-closo-dodecaboran-1-yl-phenylcarbamoyl]benzoic acid, and 4-[4,5,7,8,9,10-hexahydro-7,7,10,10-tetramethyl-1-(3-pyridylmethyl)-anthra[1,2-b]pyrrol-3-yl]benzoic acid, and (3-pyridylmethyl)-]5-thiaanthra[2,1-b]pyrrol-3-yl)benzoic acid, and (3-pyridylmethyl)-anthra[2m 1-d]pyrazol-3-yl]benzoic acid.

Representative examples of such retinoid X receptor antagonist include, without limitation, LGN100572, 1-(3-hydroxy-5,6,7,8-tetrahydro-5,5,8,8-tetramethylnaphthalene-2-yl)ethanone, 1-(3-propoxy-5,6,7,8-tetrahydro-5,5,8,8-tetramethylnaphthalene-2-yl)ethanone, 3-(3-propoxy-5,6,7,8-tetrahydro-5,5,8,8-tetramethylnaphthalene-2-yl)but-2-enenitrile, 3-(3-propoxy-5,6,7,8-tetrahydro-5,5,8,8-tetramethylnaphthalene-2-yl)but-2-enal, (2E,4E,6E)-7-3 [-propoxy-5,6,7,8-tetrahydro 5,5,8,8-tetramethyl-2-naphthalene-2-yl]-3-methylocta-2,4,6-trienoic acid, 4-[3,5,5,8,8-pentamethyl-5,6,7,8-tetrahydro-2-naphthyl)carbonyl] benzoic acid, 4-[1-(3,5, 5,8,8-pentamethyl-5,6,7,8-tetrahydro-2-naphthyl)ethenyl] benzoic acid, 4-[1 (3,5,5,8,8-pentamethyl-5,6,7,8-tetrahydro-2-naphthyl)cyclopropyl] benzoic acid, 4-[1-(3,5,5,8,8-pentamethyl-5,6,7,8-tetrahydro-2-naphthyl)ethenyl] benzenete trazole, 2-[1-(5,5,8,8-tetramethyl-5,6,7,8-tetrahydro-2-naphthyl) ethenyl]pyridine-5-carboxylic acid, 2-[1-(3,5,5,8,8-pentamethyl-5,6,7,8-tetrahydro-2-naphthyl)ethyl]pyridine-5-carboxylic acid, ethyl-2-[1-(3,5,5,8,8-pentamethyl-5,6,7,8-tetrahydro-2-naphthyl)ethenyl]pyridine-5-carboxylate, 5-[1-3,5,5,8,8-pentamethyl-5,6,7,8-tetrahydro-2-naphthyl)ethenyl]pyridine-2-carboxylic acid, 2-[1-(3,5,5,8,8-pentamethyl-5,6,7,8-tetrahydro-2-naphthyl) cyclopropyl]pyridine-5-carboxylic acid, methyl 2-[1-(3,5,5,8,8-pentamethyl-5,6,7,8-tetrahydro-2-naphthyl)cyclopropyl]pyridine-5-carboxylate, 4-[1-(3,5, 5,8,8-pentamethyl-5,6,7,8-tetrahydro-2-naphthyl)ethenyl]-N-(4-hydroxyphenyl) benzamide, 2-[1-(3,5,5,8,8-Pentamethyl-5,6,7,8-tetrahydro-2-naphthyl) ethenyl] pyridine-5-carboxylic acid, 2-[1-(3,5,5,8,8-Pentamethyl-5, 6,7,8-tetrahydro-2-naphthyl)cyclopropyl]pyridine-5-carboxylic acid, 4-[(3,5, 5,8,8-pentamethyl-5,6,7,8-tetrahydro-2-naphthyl)carbonyl]benzoic acid butyloxime, 4-[(3,5,5,8,8-pentamethyl-5,6,7,8-tetrahydro-2-naphthyl) carbonyl]benzoic acid propyloxime, 4-[(3,5,5,8,8-pentamethyl-5,6,7,8-terrahydro-2-naphthyl)carbonyl]benzoic acid cyanoimine, 4-[(3,5,5,8,8-pentamethyl-5,6,7,8-tetrahydro-2-naphthyl)carbonyl]benzoic acid allyloxime, 4-[(3,5,5,8,8-pentamethyl-5,6,7,8-tetrahydro-2-naphthyl)carbonyl]benzoic acid 4-(3-methylbut-2-enoic acid)oxime, and 4-[(3,5,5,8,8-pentamethyl-5,6,7,8-tetrahydro-2-naphthyl)carbonyl]benzoic acid 1-aminoethyloxime (2E,4E,6Z)-7-(3-n-propoxy-5,6,7,8-tetrahydro-5,5,8,8-tetramethylnaphthalene-2-yl)-3-methylocta-2,4,6-trienoic acid, and 4-(5H-2,3(2,5 dimethyl-2,5-hexano)-5-n-propyldibenzo[b,e][1,4]diazepin-11-yl)benzoic acid, and 4-(5H-2,3-(2,5-dimethyl-2,5-hexano)-5m.

Representative examples of such Vitamin D receptor antagonist include, without limitation: 1 alpha, 25-(OH)-D3-26,23 lactone; 1alpha, 25-dihydroxyvitamin D (3); the 25-carboxylic ester ZK159222; (23S)-25-dehydro-1 alpha-OH-D (3); (23R)-25-dehydro-1 alpha-OH-D (3); 1 beta, 25 (OH)₂ D₃; 1 beta, 25(OH)₂-3-epi-D₃; (23S) 25-dehydro-1 alpha(OH) D3-26,23-lactone; (23R) 25-dehydro-1 alpha(OH)D3-26,23-lactone and Butyl-(5Z,7E,22E-(1S,7E,22E-(1S,3R,24R)-1,3,24-trihydroxy-26,27-cyclo-9,10-secocholesta-5,7,10(19),22-tetraene-25-carboxylate).

The above listed antagonists are known for their high affinity towards their respective cognate receptors. However, it may be possible for these molecules to be active towards other receptors.

Hence, in another particular, the method of ex-vivo expanding a population of hematopoietic stem cells, while at the same time, substantially inhibiting differentiation of the hematopoietic stem cells ex-vivo is effected by providing hematopoietic mononuclear cells which comprise a major fraction of hematopoietic committed cells and a minor fraction of hematopoietic stem and progenitor cells, with ex-vivo culture conditions for ex-vivo cell proliferation and, at the same time, for reducing a capacity of the hematopoietic mononuclear cells in responding to signaling pathways involving PI 3-kinase, to thereby expand a population of hematopoietic stem cells, while at the same time, substantially inhibiting differentiation of the hematopoietic stem cells ex-vivo.

In another particular, the method of ex-vivo expanding a population of hematopoietic stem cells, while at the same time, substantially inhibiting differentiation of the hematopoietic stem cells ex-vivo is effected by providing hematopoietic mononuclear cells which comprise a major fraction of hematopoietic committed cells and a minor fraction of hematopoietic stem and progenitor cells, with ex-vivo culture conditions for ex-vivo cell proliferation and with nicotinamide, a nicotinamide analog, a nicotinamide or a nicotinamide analog derivative or a nicotinamide or a nicotinamide analog metabolite, to thereby expand a population of hematopoietic stem cells, while at the same time, substantially inhibiting differentiation of the hematopoietic stem cells ex-vivo.

In yet another particular, the method of ex-vivo expanding a population of hematopoietic stem cells, while at the same time, substantially inhibiting differentiation of the hematopoietic stem cells ex-vivo is effected by providing hematopoietic mononuclear cells which comprise a major fraction of hematopoietic committed cells and a minor fraction of hematopoietic stem and progenitor cells, with ex-vivo culture conditions for ex-vivo cell proliferation and with a PI 3-kinase inhibitor, to thereby expand a population of hematopoietic stem cells, while at the same time, substantially inhibiting differentiation of the hematopoietic stem cells ex-vivo.

Final concentrations of the nicotinamide or the analogs, derivatives or metabolites thereof and of the PI 3-kinase inhibitor are preferably, depending on the specific application, in the millimolar ranges. For example, within about 0.1 mM to about 20 mM, preferably within about 1 mM to about 10 mM, more preferably within about 5 mM to about 10 mM.

As is described hereinabove and is further exemplified in the Examples section that follows, expansion of the hematopoietic stem cells population present in hematopoietic mononuclear cells can also be effected in the presence of copper chelators or chalets. As is discussed in detail in WO 00/18885 and in PCT/IL03/00062, addition of copper chelators or copper chelates to cells culturing media affects the cellular copper concentration, which in turn, affects signaling pathways associated with cells differentiation. According to the teachings of WO 00/18885 and PCT/IL03/00062, addition of a copper chelate to the cells culturing media maintains the free copper concentration of the cells substantially unchanged during cell expansion, while addition of a copper chelator to the cells culturing media reduces the capacity of the cells in utilizing copper.

As used herein, the phrase “copper chelator” refers to a ligand that has at least two atoms capable of coordinating with copper or a copper ion, so as to form a ring. A copper chelator is free of, i.e., not complexed with, the copper ion. Additional features relating to chelating effects are described, for example, in PCT/IL03/00062.

As used herein throughout, the phrase “copper chelate” refers to a copper chelator, as is defined hereinabove, which is complexed with a copper ion.

Hence, according to the present invention there is provided another method of ex-vivo expanding a population of hematopoietic stem cells, while at the same time, substantially inhibiting differentiation of the hematopoietic stem cells ex-vivo. The method, according to this aspect of the present invention is effected by providing hematopoietic mononuclear cells which comprise a major fraction of hematopoietic committed cells and a minor fraction of hematopoietic stem and progenitor cells, with ex-vivo culture conditions for ex-vivo cell proliferation and with one or more copper chelator(s) or copper chelate(s), to thereby expand a population of hematopoietic stem cells, while at the same time, substantially inhibiting differentiation of the hematopoietic stem cells ex-vivo.

The copper chelate or chelators of the present invention is oftentimes capable of forming an organometallic complex with a transition metal other than copper. As metals other than copper are typically present in the cells (e.g., zinc) or can be administered to cells during therapy (e.g., platinum), it was found that copper chelates or chelators that can also interact with other metals are highly effective. Representative examples of such transition metals include, without limitation, zinc, cobalt, nickel, iron, palladium, platinum, rhodium and ruthenium.

The copper chelates of the present invention comprise a copper ion (e.g., Cu⁻¹, Cu⁺²) and one or more copper chelator(s). Preferred copper chelators according to the present invention include polyamine molecules, which can form a cyclic complex with the copper ion via two or more amine groups present in the polyamine.

Hence, the copper chelate or chelator used in the context of the different aspects and embodiments of the present invention preferably includes a polyamine chelator, namely a polymeric chain that is substituted and/or interrupted with 1-10 amine moieties, preferably 2-8 amine moieties, more preferably 4-6 amine moieties and most preferably 4 amine moieties.

The phrases “amine moiety”, “amine group” and simply “amine” are used herein to describe a —NR′R″ group or a —NR′— group, depending on its location within the molecule, where R′ and R″ are each independently hydrogen, alkyl, cycloalkyl, aryl, heteroaryl or heterocyclic, as these terms are defined hereinbelow.

The polyamine chelator can be a linear polyamine, a cyclic polyamine or a combination thereof.

A linear polyamine, according to the present invention, can be a polyamine that has a general formula 1:
HX-Am-(Y₁B₁)₁ . . . (YnBn)ₙ-ZH Formula I
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; and B₁ and Bn are each independently an alkylene chain having between 1 and 20 substituted and/or non-substituted carbon atoms, provided that at least one of X, Z, Y₁ and Yn is a —NH group and/or at least one of the carbon atoms in the alkylene chains is substituted by an amine group.

Hence, the linear polyamine, according to the present invention, is preferably comprised of one or more alkylene chains (Am, B₁ . . . Bn, in Formula 1), is interrupted by one or more heteroatoms such as S, O and N (Y₁ . . . Yn in Formula I), 20nd terminates with two such heteroatoms (X and Z in Formula I).

Alkylene chain A, as is described hereinabove, includes 1-10 substituted or non-substituted carbon atoms and is connected, at least at one end thereof, to a heteroatom (e.g., X in Formula I). Whenever there are more than one alkylene chains A (in cases where m is greater than one), only the first alkylene chain A is connected to X. However, m is preferably I and hence the linear polyamine depicted in Formula I preferably includes only one alkylene chain A.

Alkylene chain B, as is described hereinabove, includes between 1 and 20 substituted or non-substituted carbon atoms. The alkylene chain B is connected at its two ends to a heteroatom (Y₁ . . . Yn and Z in Formula I).

The preferred linear polyamine delineated in Formula I comprises between 1 and 20 alkylene chains B, denoted as B₁ . . . Bn, where “B₁ . . . Bn” is used herein to describe a plurality of alkylene chains B, namely, B₁, B₂, B₃, . . . , Bn−1 and Bn, where n equals 0-20. These alkylene chains can be the same or different. Each of B₁ . . . Bn is connected to the respective heteroatom Y₁ . . . Yn, and the last alkylene chain in the structure, Bn, is also connected to the heteroatom Z.

It should be noted that herein throughout, whenever an integer equals 0 or whenever a component of a formula is followed by the digit 0, this component is absent from the structure. For example, if n in Formula I equals 0, there is no alkylene chain B and no heteroatom Y are meant to be in the structure.

Preferably, n equals 2-10, more preferably 2-8 and most preferably 3-5. Hence, the linear polyamine depicted in Formula I preferably includes between 3 and 5 alkylene chains B, each connected to 3-5 heteroatoms Y.

The linear polyamine depicted in Formula I must include at least one amine group, as this term is defined hereinabove, preferably at least two amine groups and more preferably at least four amine groups. The amine group can be present in the structure as the heteroatoms X, Z or Y₁ . . . Yn, such that at least one of X, Z and Y₁ . . . Yn is a —NH— group, or as a substituent of one or more of the substituted carbon atoms in the alkylene chains A and B₁ . . . Bn. The presence of these amine groups is required in order to form a stable chelate with the copper ion, as is discussed hereinabove.

The alkylene chain A preferably has a general Formula II: 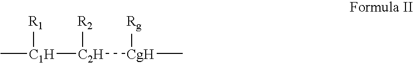
wherein g is an integer that equals 0 or 3-10.

Hence, the alkylene chain A is comprised of a plurality of carbon atoms C₁, C₂, C₃ . . . , Cg-1 and Cg, substituted by the respective R₁, R₂, R₃ . . . , Rg−1 and Rg groups. Preferably, the alkylene chain A includes 2-10 carbon atoms, more preferably, 2-6 and most preferably 2-4 carbon atoms.

As is defined hereinabove, in cases where g equals 0, the component CgH(Rg) is absent from the structure and hence the alkylene chain A comprises only 2 carbon atoms.

R₁, R₂ and Rg are each a substituent attached to the carbon atoms in A. Each of R₁, R₂ and Rg can independently be a substituent such as, but not limited to, hydrogen, alkyl, alkenyl, alkynyl, aryl, cycloalkyl, heteroalicyclic, heteroaryl, halo, amino, alkylamino, arylamino, cycloalkylamino, heteroalicyclic amino, heteroarylamino, hydroxy, alkoxy, aryloxy, azo, C-amido, N-amido, ammonium, thiohydroxy, thioalkoxy, thioaryloxy, sulfonyl, sulfinyl, N-sulfonamide, S-sulfonamide, phosphonyl, phosphinyl, phosphonium, carbonyl, thiocarbonyl, C-carboxy, O-carboxy, C-thiocarboxy, O-thiocarboxy, N-carbamate, O-carbamate, N-thiocarbamate, O-thiocarbamate, urea, thiourea, borate, borane, boroaza, silyl, siloxy, silaza, aquo, alcohol, peroxo, amine oxide, hydrazine, alkyl hydrazine, aryl hydrae, nitric oxide, cyanate, thiocyanate, isocyanate, isothiocyanate, cyano, alkylnitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, carboxylic acid, alkyl carboxylic acid, aryl carboxylic acid, sulfate, sulfite, bisulfite, thiosulfate, thiosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, phosphate, thiophosphate, phosphite, pyrophosphite, triphosphate, hydrogen phosphate, dihydrogen phosphate, guanidino, S-dithiocarbamate, N-dithiocarbamate, bicarbonate, carbonate, perchlorate, chlorate, chlorite, hypochlorite, perbromate, bromate, bromite, hypobromite, tetrahalomanganate, tetrafluoroborate, hexafluoroantimonate, hypophosphite, iodate, periodate, metaborate, tetraarylborate, tetraalkyl borate, tartarate, salicylate, succinate, citrate, ascorbate, saccharirate, amino acid, hydroxamic acid and thiotosylate.

Whenever R₁, R₂ or Rg is hydrogen, its respective carbon atom in a non-substituted carbon atom.

As used herein, the term “alkyl” is a saturated aliphatic hydrocarbon including straight chain and branched chain groups. Preferably, the alkyl group has 1 to 20 carbon atoms. More preferably, it is a medium size alkyl having 1 to 10 carbon atoms. Most preferably, it is a lower alkyl having 1 to 4 carbon atoms. The alkyl group may be substituted or non-substituted. When substituted, the substituent group can be, for example, cycloalkyl, aryl, heteroaryl, heteroalicyclic, hydroxy, alkoxy, aryloxy, thiohydroxy, thioalkoxy, thioaryloxy, cyano, halo, carbonyl, thiocarbonyl, O-carbamate, N-carbamate, O-thiocarbamate, N-thiocarbamate, C-amido, N-amido, C-carboxy, O-carboxy, nitro, sulfonamide, silyl, guanidine, urea or amino, as these terms are defined hereinbelow.

The term “alkenyl” describes an alkyl group which consists of at least two carbon atoms and at least one carbon-carbon double bond.

The term “alkynyl” describes an alkyl group which consists of at least two carbon atoms and at least one carbon-carbon triple bond.

The term “cycloalkyl” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, aryl, heteroaryl, heteroalicyclic, hydroxy, alkoxy, aryloxy, thiohydroxy, thioalkoxy, thioaryloxy, cyano, halo, carbonyl, thiocarbonyl, C-carboxy, O-carboxy, O-carbamate, N-carbamate, C-amido, N-amido, nitro, or amino, as these terms are defied hereinabove or hereinbelow.

The term “aryl” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substitute or unsubstituted. When substituted, the substituent group can be, for example, halo, trihalomethyl, alkyl, hydroxy, alkoxy, aryloxy, thiohydroxy, thiocarbonyl, C-carboxy, O-carboxy, O-carbamate, N-carbamate, O-thiocarbamate, N-thiocarbamate, C-amido, N-amido, sulfinyl, sulfonyl or amino, as these terms are defined hereinabove or hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, cycloalkyl, halo, trihalomethyl, hydroxy, alkoxy, aryloxy, thiohydroxy, thiocarbonyl, sulfonamide, C-carboxy, O-carboxy, sulfinyl, sulfonyl, O-carbamate, N-carbamate, O-thiocarbamate, N-thiocarbamate, C-amido, N-amido or amino, as these terms are defined hereinabove or hereinbelow.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, cycloalkyl, aryl, heteroaryl, halo, trihalomethyl, hydroxy, alkoxy, aryloxy, thiohydroxy, thioalkoxy, thioaryloxy, cyano, nitro, carbonyl, thiocarbonyl, C-carboxy, O-carboxy, O-carbamate, N-carbamate, O-thiocarbamate, N-thiocarbamate, sulfinyl, sulfonyl, C-amido, N-amido or amino, as these terms are defined hereinabove or hereinbelow.

The term “halo” describes a fluorine, chlorine, bromine or iodine atom.

The term “amino”, as is defined hereinabove with respect to an “amine” or an “amino group”, is used herein to describe an —NR′R″, wherein R′ and R″ are each independently hydrogen, alkyl, cycloalkyl, aryl, heteroaryl or heterocyclic, as these terms are defined hereinabove.

Hence, the terms “alkylamino”, “arylamino”, “cycloalkylamino”, “heteroalicyclic amino” and “heteroarylamino” describe an amino group, as defined hereinabove, wherein at least one of R′ and R″ thereof is alkyl, aryl, cycloalkyl, heterocyclic and heteroaryl, respectively.

The term “hydroxy” describes an —OH group.

An “alkoxy” describes both an —O-alkyl and an —O-cycloalkyl group, as defined herein.

An “aryloxy” describes both an —O-aryl and an —O-heteroaryl group, as defined herein.

The term “azo” describes a —N═N group.

A “C-amido” describes a —C(═O)—NR′R″ group, where R′ and R″ are as defined hereinabove.

An “N-amido” describes a RC(═O)—NR”— group, where R′ and R″ are as defined hereinabove.

An “ammonium” describes an —N+HR′R″ group, where R′ and R″ are as defined hereinabove.

The term “thiohydroxy” describes a —SH group.

The term “thioalkoxy” describes both a —S-alkyl group and a —S-cycloalkyl group, as defined hereinabove.

The term “thioaryloxy” describes both a —S-aryl and a —S-heteroaryl group as defined hereinabove.

A “sulfinyl” describes a —S(═O)—R group, where R can be, without limitation, alkyl, cycloalkyl, aryl and heteroaryl as these terms are defined hereinabove.

A “sulfonyl” describes a —S(═O)₂—R group, where R is as defined hereinabove.

A “S-sulfonamido” is a —S(═O)₂—NR′R″ group, with R′ and R″ as defied hereinabove.

A “N-sulfonamido” is an R′(S═O)₂—NR″— group, with R′ and R″ as defined hereinabove.

A “phosphonyl” is a —O—P(═O)(OR′)-R″ group, with R′ and R″ as defined hereinabove.

A “phosphinyl” is a —PR′R″ group, with R′ and R″ as defined hereinabove.

A “phosphonium” is a —P+R′R″R′″, where R′ and R″ are as defined hereinabove and R′″ is defined as either R′ or R″.

The term “carbonyl” describes a —C(═O)—R group, where R is hydrogen, alkyl, cycloalkyl, aryl, heteroaryl (bonded through a ring carbon) or heteroalicyclic (bonded through a ring carbon) as defined hereinabove.

A “thiocarbonyl” describes a —C(═S)—R group, where R is as defined hereinabove with respect to the term “carbonyl”.

A “C-carboxy” describes a —C(═O)—O—R groups, where R is as defined hereinabove with respect to the term “carbonyl”.

An “O-carboxy” group refers to a RC(═O)—O— group, where R is as defined hereinabove with respect to the term “carbonyl”.

A “carboxylic acid” is a C-carboxy group in which R is hydrogen.

A “C-thiocarboxy” is a —C(═S)—O—R groups, where R is as defined hereinabove with respect to the term “carbonyl”.

An “O-thiocarboxy” group refers to an R—C(═S)—O— group, where R is as defined hereinabove with respect to the term “carbonyl”.

The term “O-carbamate” describes an —OC(═O)—NR′R″ group, with R′ and R″ as defined hereinabove.

A “N-carbamate” describes a R′-O—C(═O)—NR″— group, with R′ and R″ as defined hereinabove.

An “O-thiocarbamate” describes an—C(═S)—NR′R″ group, with R′ and R″ as defined hereinabove.

A “N-thiocarbamate” describes a R′OC(═S)NR″— group, with R′ and R″ as defined hereinabove.

The term “urea” describes a —NR′-C(═O)—NR′R″ group, with R′, R″ and R′″ as defined hereinabove.

The term “thiourea” describes a —NR′-C(═S)—NR′R″ group, with R′, R″ and R′″ as defined hereinabove.

The term “borate” describes an —O—B—(OR)₂ group, with R as defined hereinabove.

The term “borane” describes a —B—R′R″ group, with R′ and R″ as defined hereinabove.

The term “boraza” describes a —B(R′)(NR″R′″) group, with R′, R″ and R′″ as defined hereinabove.

The term “silyl” describes a —SiR′R″R′″, with R′, R″ and R′″ as defined herein.

The term “siloxy” is a —Si—(OR)₃, with R as defined hereinabove.

The term “silaza” describes a —Si—(NR′R″)₃, with R′ and R″ as defined herein.

The term “aquo” describes a H₂O group.

The term “alcohol” describes a ROH group, with R as defined hereinabove.

The term “peroxo” describes an —OOR group, with R as defined hereinabove.

As used herein, an “amine oxide” is a —N(═O)R′R″R′″ group, with R′, R″ and R′″ as defined herein.

A “hydrazine” is a —NR′-NR″R′″ group, with R′, R″ and R′″ as defined herein.

Hence, “alkyl hydrazine” and “aryl hydrazine” describe a hydrazine where R′ is an alkyl or an aryl, respectively, and R″ and R′″ are as defined hereinabove.

The term “nitric oxide” is a —N═O group.

The term “cyano” is a —C═N group.

A “cyanate” is an —O—C═N group.

A “thiocyanate” is a “—S—C═N group.

An “isocyanate” is a —N═C=O group.

An “isothiocyanate” is a —N═C=S group.

The terms “alkyl nitrile” and “aryl nitrile” describe a —R—C═N group, where R is an alkyl or an aryl, respectively.

The terms “alkyl isonitrile” and “aryl isonitrile” describe a R—N═C— group, where R is an alkyl or aryl, respectively.

A “nitrate” or “nitro” is a —NO₂ group.

A “nitrite” is an —O—N═O group.

An “azido” is a N₃⁺ group.

An “alkyl sulfonic acid” and an “aryl sulfonic acid” describe a —R—SO₂—OH group, with R being an alkyl or an aryl, respectively.

An “alkyl sulfoxide”, an “aryl sulfoxide” and an “alkyl aryl sulfoxide” describe a —R′S(═O)R″ group, where R′ and R″ are each an alkyl, R′ and R″ are each an aryl and where R′ is and alkyl and R″ is an aryl, respectively.

An “alkyl sulfenic acid” and “aryl sulfenic acid” describe a —R—S—OH group, where R is an alkyl or an aryl, respectively.

An “alkyl sulfinic acid” and “aryl sulfinic acid” describe a —R—S(═O)—OH group where R is an alkyl or an aryl, respectively.

As used herein, the terms “alkyl carboxylic acid” and “aryl carboxylic acid” describe a —R—C(═O)—OH group, where R is an alkyl or an aryl, respectively.

An “alkyl thiol carboxylic acid” and an “aryl thiol carboxylic acid” describe a —R—C(═O)—SH group, where R is an alkyl or an aryl, respectively.

An “alkyl thiol thiocarboxylic acid” and an “aryl thiol thiocarboxylic acid” describe a —R—C(═S)—SH group, where R is an alkyl or an aryl, respectively.

A “sulfate” is a —O—SO₂—OR′ group, with R′ as defined hereinabove.

A “sulfite” group is a —O—S(═O)—OR′ group, with R′ as defined hereinabove.

A “bisulfite” is a sulfite group, where R′ is hydrogen.

A “thiosulfate” is an —O—SO₂—SR′ group, with R′ as defined hereinabove.

A “thiosulfite” group is an —O—S(═O)—SR′ group, with R′ as defined hereinabove.

The terms “alkyl/aryl phosphine” describe a —R—PH₂ group, with R being an alkyl or an aryl, respectively, as defined above.

The terms “alkyl and/or aryl phosphine oxide” describe a —R′-PR₂(═O) group, with R′ and R″ being an alkyl and/or an aryl, as defined hereinabove.

The terms “alkyl and/or aryl phosphine sulfide” describe a —R′-PR₂(S) group, with R′ and R″ being an alkyl and/or an aryl, as defined hereinabove.

The terms “alkyl/aryl phosphonic acid” describe a —R′-P(═O)(OH)₂ group, with R′ being an alkyl or an aryl as defined above.

The terms “alkyl/aryl phosphinic acid” describes a —R′-P(OH)₂ group, with R′ being an alkyl or an aryl as defined above.

A “phosphate” is a —O—P(═O)(OR′)(OR″) group, with R′ and R″ as defined hereinabove.

A “hydrogen phosphate” is a phosphate group, where R′ is hydrogen.

A “dihydrogen phosphate” is a phosphate group, where R′ and R″ are both hydrogen.

A “thiophosphate” is a —S—P(═O)(OR′)₂ group, with R′ as defined hereinabove.

A “phosphite” is an —O—P(OR′)₂ group, with R′ as defined hereinabove.

A “pyrophosphite” is an —O—P—(OR′)—O—P(OR″)₂ group, with R′ and R″ as defined hereinabove.

A “triphosphate” describes an —OP(═O)(OR′)-O—P(═O)(OR”)—O—P(═O)(OR′″)₂, with R′, R″ and R′″ are as defined hereinabove.

As used herein, the term “guanidine” describes a —R′NC(═N)—NR′R′″ group, with R′, R″ and R′″ as defined herein.

The tern “S-dithiocarbamate” describes a —SC(═S)—NR′R″ group, with R′ and R″ as defined hereinabove.

The term “N-dithiocarbamate” describes an R′SC(═S)—NR″— group, with R′ and R″ as defined hereinabove.

A “bicarbonate” is an —O—C(═O)—O⁻ group.

A “carbonate” is an —O—C(═O)—OH group.

A “perchlorate” is an —O-Cl(═O)₃ group.

A “chlorate” is an —O-Cl(═O)₂ group.

A “chlorite” is an —O-Cl(═O) group.

A “hypochlorite” is an —OCl group.

A “perbromate” is an —O—Br(═O)₃ group.

A “bromate” is an —O—Br(═O)₂ group.

A “bromite” is an —O—Br(═O) group.

A “hypobromite” is an —OBr group.

A “periodate” is an —O—I(═O)₃ group.

A “iodate” is an —O—I(═O)₂ group.

The term “tetrahalomanganate” describes MnCl4, MnBr₄ and MnI₄.

The term “tetrafluoroborate” describes a —BF₄ group.

A “tetrafluoroantimonate” is a SbF₆ group.

A “hypophosphite” is a —P(OH)₂ group.

The term “metaborate” describes the group 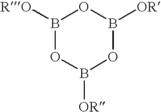
where R′, R″ and R′″ are as defined hereinabove.

The terms “tetraalkyl/tetraaryl borate” describe a R′B⁻ group, with R′ being an alkyl or an aryl, respectively, as defined above.

A “tartarate” is an —OC(═O)—CH(OH)—CH(OH)—C(═O)OH group.

A “salycilate” is the group

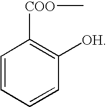

A “succinate” is an —O—C(═O)—(CH₂)₂—COOH group.

A “citrate” is an —O—C(═O)—CH₂—CH(OH)(COOH)—CH₂—COOH group.

An “ascorbate” is the group

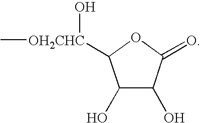

A “saccharirate” is an oxidized saccharide having two carboxylic acid group.

The term “amino acid” as used herein includes natural and modified amino acids and hence includes the 21 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids which are linked via a peptide bond or a peptide bond analog to at least one addition amino acid as this term is defined herein.

A “hydroxamic acid” is a —C(═O)—NH—OH group.

A “thiotosylate” is the group

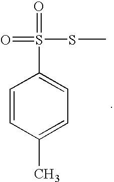

Similarly, each of the alkylene chains B₁ . . . Bn independently has a general formula III: 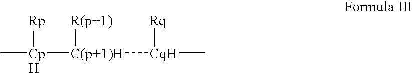
wherein p is an integer that equals 0 or g+1 and q is an integer from g+2 to g+20.

Hence, each of the alkylene chains B₁ . . . Bn is comprised of a plurality of carbon atoms Cp, Cp+1, Cp+2 . . . , Cq−1 and Cq, substituted by the respective Rp, Rp+1, Rp+2 . . . , Rq−1 and Rq groups. Preferably, each of the alkylene chains B₁ . . . Bn includes 2-20 carbon atoms, more preferably 2-10, and most preferably 2-6 carbon atoms.

As is defined hereinabove, in cases where p equals 0, the component —CpH(Rp)— is absent from the structure. In cases where p equals g+1, it can be either 1 or 4-11. The integer q can be either 2 or 5-20.

Each of the substituents Rp, Rp+1 Rn can be any of the substituents described hereinabove with respect to R₁, R₂ and Rg.

Hence, a preferred linear polyamine according to the present invention includes two or more alkylene chains. The alkylene chains are interrupted therebetween by a heteroatom and each is connected to a heteroatom at one end thereof. Preferably, each of the alkylene chains include at least two carbon atoms, so as to enable the formation of a stable chelate between the heteroatoms and the copper ion.

The linear polyamine delineated in Formula I preferably includes at least one chiral carbon atom. Hence, at least one of C₁, C₂ and Cg in the alkylene chain A and/or at least one of Cp, Cp+1 and Cq in the alkylene chain B is chiral.

A preferred linear polyamine according to the present invention is tetraethylenepentamine. Other representative examples of preferred linear polyamines usable in the context of the present invention include, without limitation, ethylendiamine, diethylenetriamine, triethylenetetramine, triethylenediamine, aminoethylethanolamine, pentaethylenehexamine, triethylenetetramine, N,N′-bis(3-aminopropyl)-1,3-propanediamine, and N,N′-Bis(2-animoethyl)-1,3 propanediamine.

In cases where the polyamine chelator is a cyclic polyamine, the polyamine can have a general formula IV: 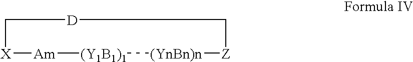
wherein m is an integer from 1 to 10; n is an integer from 0 to 20; X and Z are each independently selected from the group consisting of an oxygen atom, a sulfur atom and a —NH group; Y₁ and Yn are each independently selected from the group consisting of an oxygen atom, a sulfur atom and a —NH group; A is an alkylene chain having between 1 and 10 substituted and/or non-substituted carbon atoms; B₁ and Bn are each independently an alkylene chain having between 1 and 20 substituted and/or non-substituted carbon atoms; and D is a bridging group having a general formula V:
U-W-V Formula V
whereas U and V are each independently selected from the group consisting of substituted hydrocarbon chain and non-substituted hydrocarbon chain; and W is selected from the group consisting of amide, ether, ester, disulfide, thioether, thioester, imine and alkene, provided that at least one of said X, Z, Y₁ and Yn is a —NH group and/or at least one of said carbon atoms in said alkylene chains is substituted by an amine group.

Optionally, the cyclic polyamine has one of the general formulas VI-X: 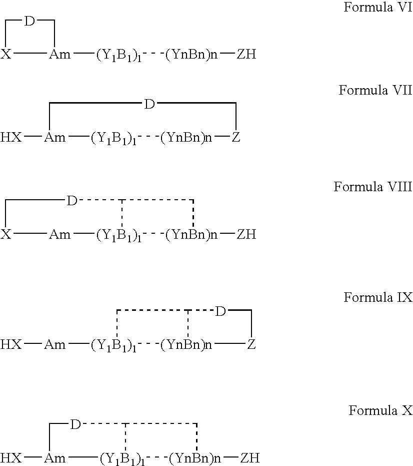
wherein m, n, X, Y₁, Yn, Z, A, B and D are as described above and further wherein should the bridging group D is attached at one end to A (Formulas VI, VII and X), U or V are being attached to one carbon atom in the alkylene chain and should D is attached at one end to B I or Bn (Formulas VIII, IX and X), U or V are being attached to one carbon atom in the alkylene chain.

Hence, a preferred cyclic polyamine according to the present invention includes two or more alkylene chains, A, B₁ . . . Bn, as is detailed hereinabove with respect to the linear polyamine. The alkylene chains can form a cyclic structure by being connected, via the bridging group D, between the ends thereof, namely between the heteroatoms X and Z (Formula IV). Optionally, the alkylene chains can form a conformationally restricted cyclic structure by being connected, via the bridging group D, therebetween (Formula X). Further optionally, a conformationally restricted cyclic structure can be formed by connecting one alkylene chain to one terminal heteroatom (X or Z, Formulas VI-IX).

As is described hereinabove, in cases where the cyclic structure is formed by connecting one alkylene chain to one terminal heteroatom, as is depicted in Formulas VI-IX, the bridging group D connects a terminal heteroatom, namely X or Z, and one carbon atom in the alkylene chains A and B₁ . . . Bn. This carbon atom can be anyone of C₁, C₂, Cg, Cp, Cp+1 and Cq described hereinabove.

As is further described hereinabove, the cyclic structure is formed by the bridging group D, which connects two components in the structure. The bridging group D has a general formula U-W-V, where each of U and V is a substituted or non-substituted hydrocarbon chain.

As used herein, the phrase “hydrocarbon chain” describes a plurality of carbon atoms which are covalently attached one to another and are substituted, inter alia, by hydrogen atoms. The hydrocarbon chain can be saturated, unsaturated, branched or unbranched and can therefore include one or more alkyl, alkenyl, alkynyl, cycloalkyl and aryl groups and combinations thereof.

The length of the hydrocarbon chains, namely the number of carbon atoms in the chains, is preferably determined by the structure of the cyclic polyamine, such that on one hand, the ring tension of the formed cyclic structure would be minimized and on the other hand, an efficient chelation with the copper ion would be achieved.

When the hydrocarbon chain is substituted, the substituents can be any one for combinations of the substituents described hereinabove with respect to R₁, R₂ and Rg in the linear polyamine.

The two hydrocarbon chains are connected therebetween by the group W, which can be amide, ether, ester, disulfide, thioether, thioester, imine and alkene.

As used herein, the term “ether” is an —O— group.

The term “ester” is a —C(═O)—O— group.

A “disulfide” is a —S—S— group.

A “thioether” is a —S— group.

A “thioester” is a —C(═O)—S— group.

An “imine” is a —C(═NH)— group.

An “alkene” is a —CH═CH— group.

The bridging group D is typically formed by connecting reactive derivatives of the hydrocarbon chains U and V, so as to produce a bond therebetween (W), via well-known techniques, as is described, for example, in U.S. Pat. No. 5,811,392.

As is described above with respect to the linear polyamine, the cyclic polyamine must include at least one amine group, preferably at least two amine groups and more preferably at least four amine groups, so as to form a stable copper chelate.

A preferred cyclic polyamine according to the present invention is cyclam (1,4,8,11-tetraazacyclotetradecane).

As is described hereinabove, the polyamine chelator of the present invention can further include a multimeric combination of one or more linear polyamine(s) and one or more cyclic polyamine(s). Such a polyamine chelator can therefore be comprised of any combinations of the linear and cyclic polyamines described hereinabove.

Preferably, such a polyamine chelator has a general Formula XI:
{(E₁)_{f}-[Q₁-(G₁)_{g}]}ₕ-{(E₂)ᵢ-[Q₂-(G₂)ⱼ]}ₖ- . . . -{(Eₙ)₁-[Qₙ-(Gₙ)]}ₜ Formula XI
wherein n is an integer greater than 1; each of f, g, h, i, j, k, l, o and t is independently an integer from 0 to 10; each of E₁, E₂ and En is independently a linear polyamine, as is described hereinabove; each of G₁, G₂ and Gn is independently a cyclic polyamine as is described hereinabove; and each of Q₁, Q₂ and Qn is independently a linker linking between two of said polyamines, provided that at least one of said Q₁, Q₂ and Qn is an amine group and/or at least one of said linear polyamine and said cyclic polyamine has at least one free amine group.

Each of E₁, E₂ and En in Formula XI represent a linear polyamine as is described in detail hereinabove, while each of G₁, G₂ and Gn represents a cyclic polyamine as is described in detail hereinabove.

The polyamine described in Formula XI can include one or more linear polyamine(s), each connected to another linear polyamine or to a cyclic polyamine.

Each of the linear or cyclic polyamines in Formula XI is connected to another polyamine via one or more linker(s), represented by Q₁, Q₂ and Qn in Formula XI.

Each of the linker(s) Q₁, Q₂ and Qn can be, for example, alkylene, alkenylene, alkynylene, arylene, cycloalkylene, hetroarylene, amine, azo, amide, sulfonyl, sulfinyl, sulfonamide, phosphonyl, phosphinyl, phosphonium, ketoester, carbonyl, thiocarbonyl, ester, ether, thioether, carbamate, thiocarbamate, urea, thiourea, borate, borane, boroaza, silyl, siloxy and silaza.

As used herein, the term “alkenylene” describes an alkyl group which consists of at least two carbon atoms and at least one carbon-carbon double bond.

The term “alkynylene” describes an alkyl group which consists of at least two carbon atoms and at least one carbon-carbon triple bond.

The term “cycloalkylene” describes an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane.

The term “arylene” describes an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted.

The term “heteroarylene” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

As used in the context of the linker of the present invention, the term “amine” describes an —NR′-, wherein R′ can be hydrogen, alkyl, cycloalkyl, aryl, heteroaryl or heterocyclic, as these terms are defined hereinabove.

As is further used in the context of the linker of the present invention, the term “azo” describes a —N═N— group.

The term “amide” describes a —C(═O)—NR′— group, where R′ is as defined hereinabove.

The term “ammonium” describes an —N⁺HR′— group, where R′ is as defined hereinabove.

The term “sulfinyl” describes a —S(═O)— group.

The term “sulfonyl” describes a —S(═O)₂— group.

The term “sulfonamido” describes a —S(═O)₂—NR′— group, with R′ as defined hereinabove.

The term “phosphonyl” describes a —O—P(═O)(OR′)-group, with R′ as defined hereinabove.

The term “phosphinyl” describes a —PR′— group, with R′ as defined hereinabove.

The term “phosphonium” is a —P⁺R′R″, where R′ and R″ are as defined hereinabove.

The term “ketoester” describes a —C(═O)—C(═O)—O— group.

The term “carbonyl” describes a —C(═O)— group.

The term “thiocarbonyl” describes a —C(═S)— group.

The term “carbamate” describes an —OC(═O)—NR′— group, with R′ as defined hereinabove.

The term “thiocarbamate” describes an —OC(═S)—NR— group, with R′ as defined hereinabove.

The term “urea” describes an —NR′—C(═O)—NR″— group, with R′ and R″ and as defined hereinabove.

The term “thiourea” describes a —NR′—C(═S)—NR′— group, with R′ and R″ as defined hereinabove.

The term “borate” describes an —O—B—(OR)— group, with R as defined hereinabove.

The term “borane” describes a —B—R—′— group, with R as defined hereinabove.

The term “boraza” describes a —B (NR′R″)— group, with R′ and R″ as defined hereinabove.

The term “silyl” describes a —SiR′R″—, with R′ and R″ as defined herein.

The term “siloxy” is a —Si—(OR)₂—, with R as defined hereinabove.

The term “silaza” describes a —Si—(NR′R″)₂—, with R′ and R″ as defined herein.

It should be noted that all the terms described hereinabove in the context of the linker of the present invention are the same as described above with respect to the substituents. However, in distinction from the substituent groups, which are connected to a component at one end thereof, the linker groups are connected to two components at two sites thereof and hence, these terms have been redefined with respect to the linker.

As has been mentioned hereinabove, according to the presently most preferred embodiment of the present invention, the polyamine chelator 25 is tetraethylenepentamine (TEPA). However, other preferred polyamine chelators include, without limitation, ethylendiamine, diethylenetriamine, triethylenetetramine, triethylenediamine, aminoethylethanolamine, aminoethylpiperazine, pentaethylenehexamine, triethylenetetramine, captopril, penicilamine, N,N′-bis(3-aminopropyl)-1,3-propanediamine, N,N′-B is(2-animoethyl)-1,3-propanediamine, 1,7-dioxa-4,10-diazacyclododecane, 1,4,8,11-tetraazacyclotetradecane-5,7-dione, 1,4,7-triazacyclononane, 1-oxa-4,7, 10-triazacyclododecane, 1,4,8,12-tetraazacyclopentadecane and 1,4,7,10-tetraazacyclododecane.

The above listed preferred chelators are known in their high affinity towards copper ions. However, these chelators are further beneficially characterized by their substantial affinity also towards other transition metals, as is described by Ross and Frant [Ross J W and Frant M S. Chelometric indicators, titration with the solid-state cupric ion selective electrode. Analytical Chemistry 41:1900, 1969], which is incorporated by reference as if fully set forth herein.

All the polyamine chelators described hereinabove can be either commercially obtained or can be synthesized using known procedures such as described, for example, in: T. W. Greene (ed.), 1999 (“Protective Groups in Organic Synthesis” 3ed Edition, John Wiley & Sons, Inc., New York 779 pp); or in: R. C. Larock and V. C. H. Wioley, “Comprehensive Organic Transformations—A Guide to Functional Group Preparations”, (1999) 2^{nd} Edition.

A preferred procedure for preparing tetraethylenepentamine-copper chelate (TEPA-Cu) is described in PCT/IL03/00062.

The copper chelate or chelator can be provided to the cell culture medium. The final concentrations of copper chelate may be, depending on the specific application, in the micromolar or millimolar ranges, for example, within about 0.1 μM to about 100 mM, preferably within about 4 μM to about 50 mM, more preferably within about 5 μM to about 40 mM.

The methods described hereinabove for ex-vivo expanding hematopoietic stem cell populations result, inter alia, in an expanded population of hematopoietic stem cells.

Thus, further according to an aspect of the present invention there are provided ex-vivo expanded populations of hematopoietic stem cells, obtained by any of the methods described hereinabove. The expanded populations of hematopoietic stem cells according to the present invention comprise a plurality of cells characterized by 3-20% of the cells being reselectable CD34+ cells, of which at least 40% of cells are CD34+_{dim}, i.e., fall below the median intensity in a FACS analysis, wherein, in the reselectable CD34+ cells, a majority of cells which are Lin⁻ are also CD34+_{dim} cells.

In one embodiment, the population of hematopoietic stem cells has a single genetic background.

In another embodiment, the ex-vivo expanded population of hematopoietic stem cells comprises at least N cells derived from a single donor, wherein N equals the average number of CD34+ cells derived from one sample of hematopoietic mononuclear cells, multiplied by 1,000.

Cell surface expression of the CD34 and/or Lin markers can be determined, for example, via FACS analysis or immunohistological staining techniques. A self renewal potential of the hematopoietic stem cells can be determined in-vitro by long term colony formation (LTC-CFUc), as is further exemplified in the Examples section that follows.

As is discussed in detail hereinabove, ex-vivo expansion of hematopoietic stem cells can be advantageously utilized in various applications such as, for example, hematopoietic cells transplantation or implantation, adoptive immunotherapy and gene therapy. The ability to practice the ex-vivo expansion of hematopoietic stem cells with hematopoietic mononuclear cells as the cells source substantially facilitates the utilization of the methods described hereinabove in these applications.

Hence, according to another aspect of the present invention there is provided a method of hematopoietic cells transplantation or implantation. The method according to this aspect of the present invention is effected by (a) obtaining hematopoietic mononuclear cells which comprise a major fraction of hematopoietic committed cells and a minor fraction of hematopoietic stem and progenitor cells from a donor, (b) providing the hematopoietic mononuclear cells with ex-vivo culture conditions for cell proliferation and, at the same time, for reducing an expression and/or activity of CD38, so as to expand a population of the hematopoietic stem cells, while at the same time, substantially inhibiting differentiation of the hematopoietic stem cells ex-vivo, and (c) transplanting or implanting the thus obtained hematopoietic stem cells to a recipient.

As is described hereinabove, various agents can be used in the context of the different aspects of the present invention for reducing an expression and/or activity of CD38.

Thus, in a particular embodiment of this aspect of the present invention, the method is effected by providing the hematopoietic mononuclear cells with ex-vivo culture conditions for cell proliferation and, at the same time, for reducing a capacity of the hematopoietic mononuclear cells in responding to retinoic acid, retinoids and/or Vitamin D, as is described hereinabove.

In another particular embodiment, the method is effected by providing the hematopoietic mononuclear cells with ex-vivo culture conditions for cell proliferation and, at the same time, for reducing a capacity of the hematopoietic mononuclear cells in responding to signaling pathways involving the retinoic acid receptor, the retinoid X receptor and/or the Vitamin D receptor, as is described hereinabove.

In another particular embodiment of this aspect of the present invention, the method is effected by providing the hematopoietic mononuclear cells with ex-vivo culture conditions for cell proliferation and, at the same time, for reducing a capacity of the hematopoietic mononuclear cells in responding to signaling pathways involving PI 3-kinase, as is described hereinabove.

In still another particular embodiment of this aspect of the present invention, the method is effected by providing the hematopoietic mononuclear cells with ex-vivo culture conditions for cell proliferation and with nicotinamide, a nicotinamide analog, a nicotinamide or a nicotinamide analog derivative or a nicotinamide or a nicotinamide analog metabolite, as is described hereinabove.

In another particular embodiment of this aspect of the present invention, the method is effected by providing the hematopoietic mononuclear cells with ex-vivo culture conditions for cell proliferation and with a PI 3-kinase inhibitor, as is described hereinabove.

In another aspect of the present invention, the method of hematopoietic cells transplantation or implantation described above is effected by providing the hematopoietic mononuclear cells with ex-vivo culture conditions for cell proliferation and with one or more of the copper chelator(s) or chelate(s) described hereinabove.

In any of the methods of this aspect of the present invention, the donor and the recipient can be a single individual or different individuals, for example, allogene is or xenogeneic individuals. When allogeneic transplantation is practiced, regimes for reducing implant rejection and/or graft vs. host disease, as well know in the art, should be undertaken. Such regimes are currently practiced in human therapy. Most advanced regimes are disclosed in publications by Slavin S. et al., e.g., J Clin Immunol (2002) 22: 64, and J Hematother Stem Cell Res (2002) 11: 265), Gur H et al. (Blood (2002) 99: 4174), and Martelli M F et al, (Semin Hematol (2002) 39: 48), which are incorporated herein by reference.

The methods described hereinabove can be utilized to produce transplantable hematopoietic cell preparations, such that according to yet another aspect of the present invention there is provided a transplantable hematopoietic cell preparation, which comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of an effective amount of an agent for reducing the expression and/or activity of CD38, while at the same time, substantially inhibiting differentiation of said hematopoietic stem cells, and a pharmaceutically acceptable carrier.

As is described hereinabove, various agents were found to reduce the expression and/or activity of CD38, while at the same time, substantially inhibit differentiation of the hematopoietic stem cells under these conditions.

Hence, in a particular embodiment of this aspect of the present invention, the agent described above is an agent that reduces a capacity of the hematopoietic mononuclear cells in responding to retinoic acid, retinoids and/or Vitamin D, while at the same time, substantially inhibits differentiation of the hematopoietic stem cells.

In another particular embodiment of this aspect of the present invention, the agent described above is an agent that reduces a capacity of the hematopoietic mononuclear cells in responding to retinoic acid receptor, retinoid X receptor and/or Vitamin D receptor signaling, while at the same time, substantially inhibits differentiation of the stem cells.

In yet another particular embodiment of this aspect of the present invention, the agent described above is an agent that reduces a capacity of the hematopoietic mononuclear cells in responding to PI 3-kinase signaling, while at the same time, substantially inhibits differentiation of the stem cells.

In still another particular embodiment of this aspect of the present invention, the agent described above comprises an effective amount of an agent selected from the group consisting of nicotinamide, a nicotinamide analog, a nicotinamide or a nicotinamide analog derivative and a nicotinamide or a nicotinamide analog metabolite.

In still another particular embodiment of this aspect of the present invention, the agent described above comprises an effective amount of a PI 3-kinase inhibitor.

According to still another aspect of the present invention there is provided a transplantable hematopoietic cell preparation, which comprises an expanded population of hematopoietic stem cells propagated ex-vivo from hematopoietic mononuclear cells which comprise, prior to expansion, a major fraction of hematopoietic committed cells and a minor fraction of hematopoietic stem and progenitor cells, in the presence of at least one copper chelate or chelator, as defined hereinabove, while at the same time, substantially inhibiting differentiation of said hematopoietic stem cells, and a pharmaceutically acceptable carrier.

As is further discussed hereinabove, the ex-vivo expansion of hematopoietic stem cells of the present invention can be utilized in adoptive immunotherapy.

Similarly to the hematopoietic transplantation or implementation methods of the present invention, a method of adoptive therapy according to the present invention is effected by (a) obtaining hematopoietic mononuclear cells which comprise a major fraction of hematopoietic committed cells and a minor fraction of hematopoietic stem and progenitor cells from a recipient; (b) providing the hematopoietic mononuclear cells with ex-vivo culture conditions for cell proliferation and, at the same time, with each of the copper chelators or chelates described hereinabove and/or each of the agents for reducing the expression and/or activity of CD38 described hereinabove, so as to expand the population of the hematopoietic stem cells, while at the same time, substantially inhibiting differentiation of the hematopoietic stem cells, as is detailed hereinabove; and (c) transplanting the thus obtained hematopoietic stem cells to the recipient.

As is further detailed below, stem cells in general and hematopoietic stem cells in particular may serve to exert cellular gene therapy.

Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. For review see, in general the text “Gene Therapy” (Advanced in Pharmacology 40, Academic Press, 1997).

Two basic approaches to gene therapy have evolved: (i) ex-vivo or cellular gene therapy; and (ii) in vivo gene therapy. In ex-vivo gene therapy cells are removed from a patient, and while being cultured are treated in-vitro. Generally, a functional replacement gene is introduced into the cells via an appropriate gene delivery vehicle/method (transfection, transduction, homologous recombination, etc.) and an expression system as needed and then the modified cells are expanded in culture and returned to the host/patient. These genetically re-implanted cells have been shown so express the transfected genetic material in situ.

Hence, further according to an aspect of the present invention, there is provided a method of genetically modifying stem cells with an exogene. The method, according to this aspect of the present invention, is effected by (a) obtaining hematopoietic mononuclear cells which comprise a major fraction of hematopoietic committed cells and a minor fraction of hematopoietic stem and progenitor cells, (b) providing the hematopoietic mononuclear cells with ex-vivo culture conditions for cell proliferation and, at the same time, with each of the copper chelators or chelates described hereinabove and/or each of the agents for reducing the expression and/or activity of CD38 described hereinabove, so as to expand the population of the hematopoietic stem cells, while at the same time, substantially inhibiting differentiation of the hematopoietic stem cells, as is detailed hereinabove, and (c) genetically modifying the hematopoietic stem cells with the exogene.

In a preferred embodiment, genetically modifying the cells is effected by a vector, which comprises the exogene or transgene, which vector is, for example, a viral vector or a nucleic acid vector. Many viral vectors suitable for use in cellular gene therapy are known, examples are provided hereinbelow. Similarly, a range of nucleic acid vectors can be used to genetically transform the expanded cells of the invention, as is further described below.

Accordingly, the expanded cells of the present invention can be modified to express a gene product. As used herein, the phrase “gene product” refers to proteins, peptides and functional RNA molecules. Generally, the gene product encoded by the nucleic acid molecule is the desired gene product to be supplied to a subject. Examples of such gene products include proteins, peptides, glycoproteins and lipoproteins normally produced by an organ of the recipient subject. For example, gene products which may be supplied by way of gene replacement to defective organs in the pancreas include insulin, amylase, protease, lipase, trypsinogen, chymotrypsinogen, carboxypeptidase, ribonuclease, deoxyribonuclease, triaclyglycerol lipase, phospholipase A₂, elastase, and amylase; gene products normally produced by the liver include blood clotting factors such as blood clotting Factor VIII and Factor IX, UDP glucuronyl transferae, ornithine transcarbanoylase, and cytochrome p450 enzymes, and adenosine deaminase, for the processing of serum adenosine or the endocytosis of low density lipoproteins; gene products produced by the thymus include serum thymic factor, thymic humoral factor, thymopoietin, and thymosin al; gene products produced by the digestive tract cells include gastrin, secretin, cholecystokinin, somatostatin, serotinin, and substance P.

Alternatively, the encoded gene product is one, which induces the expression of the desired gene product by the cell (e.g., the introduced genetic material encodes a transcription factor, which induces the transcription of the gene product to be supplied to the subject).

In still another embodiment, the recombinant gene can provide a heterologous protein, e.g., not native to the cell in which it is expressed. For instance, various human MHC components can be provided to non-human cells to support engraftment in a human recipient. Alternatively, the transgene is one, which inhibits the expression or action of a donor MHC gene product.

A nucleic acid molecule introduced into a cell is in a form suitable for expression in the cell of the gene product encoded by the nucleic acid. Accordingly, the nucleic acid molecule includes coding and regulatory sequences required for transcription of a gene (or portion thereof) and, when the gene product is a protein or peptide, translation of the gene acid molecule include promoters, enhancers and polyadenylation signals, as well as sequences necessary for transport of an encoded protein or peptide, for example N-terminal signal sequences for transport of proteins or peptides to the surface of the cell or secretion.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements, which are known in the art, include upstream regions from the dystrophin gene (Klamut et al., (1989) Mol. Cell Biol.9: 2396), the creatine kinase gene (Buskin and Hauschka, (1989) Mol. Cell Biol. 9: 2627) and the troponin gene (Mar and Ordahl, (1988) Proc. Natl. Acad. Sci. USA. 85: 6404). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters).

Alternatively, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

Alternatively, a regulatory element, which provides inducible expression of a gene linked thereto, can be used. The use of an inducible regulatory element (e.g., an inducible promoter) allows for modulation of the production of the gene product in the cell. Examples of potentially useful inducible regulatory systems for uses in eukaryotic cells include hormone-regulated elements (e.g., see Mader, S. and White, J. H. (1993) Proc. Natl. Acad. Sci. USA 90: 5603-5607), synthetic ligand-regulated elements (see, e.g., Spencer, D. M. et al. 1993) Science 262: 1019-1024) and ionizing radiation-regulated elements (e.g., see Manome, Y. Et al. (1993) Biochemistry 32: 10607-10613; Datta, R. et al. (1992) Proc. Natl. Acad. Sci. USA 89: 1014-1013). Additional tissue-specific or inducible regulatory systems, which may be developed, can also be used in accordance with the invention.

There are a number of techniques known in the art for introducing genetic material into a cell that can be applied to modify a cell of the invention.

In one embodiment, the nucleic acid is in the form of a naked nucleic acid molecule. In this situation, the nucleic acid molecule introduced into a cell to be modified consists only of the nucleic acid encoding the gene product and the necessary regulatory elements.

Alternatively, the nucleic acid encoding the gene product (including the necessary regulatory elements) is contained within a plasmid vector. Example of plasmid expression vectors include CDM8 (Seed, B. (1987) Nature 329: 840) and pMT2PC (Kaufman, et al. (1987) EMBO J. 6: 187-195).

In another embodiment, the nucleic acid molecule to be introduced into a cell is contained within a viral vector. In this situation, the nucleic acid encoding the gene product is inserted into the viral genome (or partial viral genome). The regulatory elements directing the expression of the gene product can be included with the nucleic acid inserted into the viral genome (i.e., linked to the gene inserted into the vial genome) or can be provided by the viral genome itself.

Naked nucleic acids can be introduced into cells using calcium phosphate mediated transfection, DEAE-dextran mediated transfection, electroporation, liposome-mediated transfection, direct injection, and receptor-mediated uptake.

Naked nucleic acid, e.g., DNA, can be introduced into cells by forming a precipitate containing the nucleic acid and calcium phosphate. For example, a HEPES-buffered saline solution can be mixed with a solution containing calcium chloride and nucleic acid to form a precipitate and the precipitate is then incubated with cells. A glycerol or dimethyl sulfoxide shock step can be added to increase the amount of nucleic acid taken up by certain cells. CaPO₄-mediated transfection can be used to stably (or transiently) transfect cells and is only applicable to in vitro modification of cells. Protocols for CaPO₄-mediated transfection can be found in Current Protocols in Molecular Biology, Ausubel, F. M. et al. (eds.) Greene Publishing Associates, (1989), Section 9.1 and in Molecular Cloning: A Laboratory Manual, 2nd Edition, Sambrook et al. Cold Spring Harbor Laboratory Press, (1989), Sections 16.32-16.40 or other standard laboratory manuals.

Naked nucleic acid can be introduced into cells by forming a mixture of the nucleic acid and DEAE-dextran and incubating the mixture with the cells. A dimethylsulfoxide or chloroquine shock step can be added to increase the amount of nucleic acid uptake. DEAE-dextran transfection is only applicable to in vitro modification of cells and can be used to introduce DNA transiently into cells but is not preferred for creating stably transfected cells. Thus, this method can be used for short-term production of a gene product but is not a method of choice for long-term production of a gene product. Protocols for DEAE-dextran-mediated transfection can be found in Current Protocols in Molecular Biology, Ausubel, F. M. et al. (eds.) Greene Publishing Associates (1989), Section 9.2 and in Molecular Cloning: A Laboratory Manual, 2nd Edition, Sambrook et al. Cold Spring Harbor Laboratory Press, (1989), Sections 16.41-16.46 or other standard laboratory manuals.

Naked nucleic acid can also be introduced into cells by incubating the cells and the nucleic acid together in an appropriate buffer and subjecting the cells to a high-voltage electric pulse. The efficiency with which nucleic acid is introduced into cells by electroporation is influenced by the strength of the applied field, the length of the electric pulse, the temperature, the conformation and concentration of the DNA and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel F. M. et al. (eds.) Greene Publishing Associates, (1989), Section 9.3 and in Molecular Cloning: A Laboratory Manual, 2nd Edition, Sambrook et al. Cold Spring Harbor Laboratory Press, (1989), Sections 16.54-16.55 or other standard laboratory manuals.

Another method by which naked nucleic acid can be introduced into cells includes liposome-mediated transfection (lipofection). The nucleic acid is mixed with a liposome suspension containing cationic lipids. The DNA/liposome complex is then incubated with cells. Liposome mediated transfection can be used to stably (or transiently) transfect cells in culture in vitro. Protocols can be found in Current Protocols in Molecular Biology, Ausubel F. M. et al. (eds.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. Natl. Acad. Sci. USA 84:7851-7855; Brigham et al. (1989) Am. J. Med. Sci. 298:278; and Gould-Fogerite et al. (1989) Gene 84:429-438.

Naked nucleic acid can also be introduced into cells by directly injecting the nucleic acid into the cells. For an in vitro culture of cells, DNA can be introduced by microinjection. Since each cell is microinjected individually, this approach is very labor intensive when modifying large numbers of cells. However, a situation wherein microinjection is a method of choice is in the production of transgenic animals (discussed in greater detail below). In this situation, the DNA is stably introduced into a fertilized oocyte, which is then allowed to develop into an animal. The resultant animal contains cells carrying the DNA introduced into the oocyte. Direct injection has also been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo can be used. Such an apparatus is commercially available (e.g., from BioRad).

Naked nucleic acid can be complexed to a cation, such as polylysine, which is coupled to a ligand for a cell-surface receptor to be taken up by receptor-mediated endocytosis (see for example Wu, G. and Wu, C. H. (1988) J. Biol. Chem. 263: 1462; Wilson et al. (1992) J. Biol. Chem. 267: 963-967; and U.S. Pat. No. 5,166,320). Binding of the nucleic acid-ligand complex to the receptor facilitates uptake of the DNA by receptor-mediated endocytosis. Receptors to which a DNA-ligand complex has targeted include the transferrin receptor and the asialoglycoprotein receptor. A DNA-ligand complex linked to adenovirus capsids which naturally disrupt endosomes, thereby releasing material into the cytoplasm can be used to avoid degradation of the complex by intracellular lysosomes (see for example Curiel et al. (1991) Proc. Natl. Acad. Sci. USA 88: 8850; Cristiano et al. (1993) Proc. Natl. Acad. Sci. USA 90: 2122-2126). Receptor-mediated DNA uptake can be used to introduce DNA into cells either in vitro or in vivo and, additionally, has the added feature that DNA can be selectively targeted to a particular cell type by use of a ligand which binds to a receptor selectively expressed on a target cell of interest.

Generally, when naked DNA is introduced into cells in culture (e.g., by one of the transfection techniques described above) only a small fraction of cells (about 1 out of 10⁵) typically integrate the transfected DNA into their genomes (i.e., the DNA is maintained in the cell episomally). Thus, in order to identify cells, which have taken up exogenous DNA, it is advantageous to transfect nucleic acid encoding a selectable marker into the cell along with the nucleic acid(s) of interest. Preferred selectable markers include those, which confer resistance to drugs such as G418, hygromycin and methotrexate. Selectable markers may be introduced on the same plasmid as the gene(s) of interest or may be introduced on a separate plasmid.

A preferred approach for introducing nucleic acid encoding a gene product into a cell is by use of a viral vector containing nucleic acid, e.g., a cDNA, encoding the gene product. Infection of cells with a viral vector has the advantage that a large proportion of cells receive the nucleic acid which can obviate the need for selection of cells which have received the nucleic acid. Additionally, molecules encoded within the viral vector, e.g., a cDNA contained in the viral vector, are expressed efficiently in cells which have taken up viral vector nucleic acid and viral vector systems can be used either in vitro or in vivo.

Defective retroviruses are well characterized for use in gene transfer for gene therapy purposes (for review see Miller, A. D. (1990) Blood 76: 271). A recombinant retrovirus can be constructed having a nucleic acid encoding a gene product of interest inserted into the retroviral genome. Additionally, portions of the retroviral genome can be removed to render the retrovirus replication defective. The replication defective retrovirus is then packaged into virions, which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM, which are well known to those skilled in the art. Examples of suitable packaging virus lines include ψCrip, ψCrip, ψ2 and ψAm. Retroviruses have been used to introduce a variety of genes into many different cell types, including epithelial cells endothelial cells, lymphocytes, myoblasts, hepatocytes, bone marrow cells, in vitro and/or in vivo (see for example Eglitis, et al. (1985) Science 230: 1395-1398; Danosand Mulligan (1988) Proc. Natl. Acad. Sci. USA 85: 6460-6464; Wilson et al. (1988) Proc. Natl. Acad. Sci USA 85:3014-3018; Ammentano et al., (1990) Proc. Natl. Acad. Sci. USA 87: 6141-6145; Huber et al. (1991) Proc. Natl. Acad. Sci. USA 88: 8039-8043; Feri et al. (1991) Proc. Natl. Acad. Sci. USA 88:8377-8381; Chowdhury et al. (1991) Science 254: 1802-1805; van Beusechem et al. (1992) Proc. Natl. Acad. Sci USA 89:7640-7644; Kay et al. (1992) Human Gene Therapy 3:641-647; Dai et al. (1992) Proc. Natl. Acad. Sci. USA 89:10892-10895; Hwu et al. (1993) J. 1 mmol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

The genome of an adenovirus can be manipulated such that it encodes and expresses a gene product of interest but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89: 6482-6486), hepatocytes (Herz and Gerard (1993) Proc. Natl. Acad. Sci. USA 90: 2812-2816) and muscle cells (Quantin et al. (1992) Proc. Natl. Acad. Sci. USA 89: 2581-2584). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity ofishe adenoviral genome for foreign DNA is large (up to 8 kilobases) relative to other gene delivery vectors (Berkner et al. cited supra; Haj-Ahmand and Graham (1986) J. Virol 57: 267). Most replication-defective adenoviral vectors currently in use are deleted for all or parts of the viral E1 and E3 genes but retain as much as 80% of the adenoviral genetic material.

Adeno-associated virus (AAV) is a naturally occurring defective virus that requires another virus, such as an adenovirus or a herpes virus, as a helper virus for efficient replication and a productive life cycle. (For a review see Muzyczka et al. Curr. Topics In Micro. And Immunol. (1992) 158: 97-129). It is also one of the few viruses that may integrate its DNA into non-dividing cells, and exhibits a high frequency of stable integration (see for example Flotte et al. (1992) Am. J. Respir. Cell. Mol. Biol. 7: 349-356; Samulski et al. (1989) J. Virol. 63:3822-3828; and McLaughlin et al. (1989) J. Virol. 62: 1963-1973). Vectors containing as little as 300 base pairs of AAV can be packaged and can integrate. Space for exogenous DNA is limited to about 4.5 kb. An AAV vector such as that described in Tratschin et oal. (1985) Mol. Cell. Biol. 5: 3251-3260 can be used to introduce DNA into cells. A variety of nucleic acids have been introduced into different cell types using AAV vectors (see for example Hermonat et al. (1984) Proc. Natl. Acad. Sci. USA 81: 6466-6470; Tratschin et al. (1985) Mol. Cell Biol. 4: 2072-2081; Wondisford et al. (1988) Mol. Endocrinol. 2:32-39; Tratschin et al. (1984) J. Virol. 51: 611-619; and Flotte et al. (1993) J. Biol. Chem. 268: 3781-3790).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone.

When the method used to introduce nucleic acid into a population of cells results in modification of a large proportion of the cells and efficient expression of the gene product by the cells (e.g., as is often the case when using a viral expression vector), the modified population of cells may be used without further isolation or subcloning of individual cells within the population. That is, there may be sufficient production of the gene product by the population of cells such that no further cell isolation is needed. Alternatively, it may be desirable to grow a homogenous population of identically modified cells from a single modified cell to isolate cells, which efficiently express the gene product. Such a population of uniform cells can be prepared by isolating a single modified cell by limiting dilution cloning followed by expanding the single cell in culture into a clonal population of cells by standard techniques.

According to a preferred embodiment of the present invention, in each of the methods described hereinabove, providing the hematopoietic mononuclear cells with conditions for ex-vivo cell proliferation is effected by providing the cells with nutrients and with cytokines. Preferably, the cytokines are early acting cytokines, such as, but not limited to, stem cell factor, FLT3 ligand, interleukin-1, interleukin-2, interleukin-3, interleukin-6, interleukin-10, interleukin-12, tumor necrosis factor-α and thrombopoietin. It will be appreciated in this respect that novel cytokines are continuously discovered, some of which may find uses in the methods of cell expansion of the present invention.

Late acting cytokines can also be used. These include, for example, granulocyte colony stimulating factor, granulocyte/macrophage colony stimulating factor, erythropoietin, FGF, EGF, NGF, VEGF, LIF, Hepatocyte growth factor and macrophage colony stimulating factor.

The ability of the agents of the present invention to inhibit differentiation of hematopoietic stem cells present in hematopoietic mononuclear cells can be further used in technical applications such as cells collection and cells culturing.

According to a further aspect of the present invention there is provided a hematopoietic stem cells collection/culturing bag. The cells collection/culturing bag of the present invention is supplemented with an effective amount of a retinoic acid receptor antagonist, a retinoid X receptor antagonist and/or a Vitamin D receptor antagonist, which substantially inhibits cell differentiation of a hematopoietic stem cells fraction of hematopoietic mononuclear cells which comprise a major fraction of hematopoietic committed cells and a minor fraction of hematopoietic stem and progenitor cells. Alternatively, the hematopoietic stem cells collection/culturing bag of the present invention is supplemented with an effective amount of nicotinamide, a nicotinamide analog, a nicotinamide or a nicotinamide analog derivative or a nicotinamide or a nicotinamide analog metabolite. Still alternatively, the hematopoietic stem cells collection/culturing bag of the present invention is supplemented with an effective amount of a PI 3-kinase inhibitor. Further alternatively, the hematopoietic stem cells collection/culturing bag of the present invention is supplemented with an effective amount of one or more copper chelator(s) or chelate(s).

According to an additional aspect of the present invention, there is provided an assay of determining whether a specific molecule/agent, e.g., a retinoic acid receptor antagonist, a retinoid X receptor antagonist, a Vitamin D receptor antagonist, a CD38 inhibitor, a PI 3-kinase inhibitor, a copper chelator or a copper chelate, is an effective agent for expanding a population of hematopoietic stem cells that are present in a hematopoietic mononuclear cells fraction.

The assay, according to this aspect of the present invention, is performed by culturing hematopoietic mononuclear cells which comprise a major fraction of hematopoietic committed cells and a minor fraction of hematopoietic stem and progenitor cells in the presence of the tested agent/molecule and monitoring expansion of the hematopoietic stem cells over time, e.g., a few weeks to a few months. If increased expansion and decreased differentiation occurs, as compared to non-treated cells, the tested agent/molecule is an effective hematopoietic stem cell expansion agent.

Preferably, culturing the hematopoietic mononuclear cells is performed in a presence of an effective amount of a cytokine, preferably, an early acting cytokine or a combination of such cytokines, e.g., thrombopoietin (TPO), interleukin-6 (IL-6), an FLT-3 ligand and stem cell factor (SCF). This assay can be used, by one ordinarily skilled in the art, to determine, for example, which of the antagonists, inhibitors or copper chelators and chelates listed above is most efficient for the purpose of implementing the various methods and preparations of the present invention described hereinabove. The assay can be further used to determine most effective concentrations and exposure time for achieving optimal results with hematopoietic mononuclear cells of different origins.

In each of the aspects of the present invention described hereinabove, the hematopoietic mononuclear cells can be obtained from any multicellular organism including both animals and plants. Preferably, the hematopoietic mononuclear cells are obtained from the bone marrow (Rowley S D et al. (1998) Bone Marrow Transplant 21: 1253), the peripheral blood (Koizumi K, (2000) Bone Marrow Transplant 26: 787, the liver (Petersen B E et al. (1998) Hepatology 27: 433) and neonatal umbilical cord blood.

Additional objects, advantages, and novel features of the present inventior will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### EXAMPLE 1

### The Effect of a Copper Chelator on the Ex-Vivo Expansion of Hema Topoietic Stem Cells of a Mononuclear Cells Culture

### Experimental Procedures

Sample collection and processing: Samples were obtained from umbilical cord blood after a normal full-term delivery and were frozen within 24 hours pospartum. The blood cells were thawed in Dextran buffer and incubated for 15 hours in MEM (Biological Industries, Israel) supplemented with 10% fetal calf serum (FCS; Biological Industries). The cells were then layered on Ficoll-Hypaque (density 1.077 gram/ml; Sigma) and centrifuged at 400 g for 30 minutes at room temperature. The mononuclear cells in the interface layer were then collected, washed three times in phosphate-buffered saline (PBS; Biological Industries), and re-suspended in PBS containing 0.5% human serum albumin (HSA). The cells were then split into two fractions, the first being the mononuclear cells (MNC) fraction and the second fraction was used for purifying CD34⁺ cells by immunomagnetic separation using the “MiniMACS CD34⁺ progenitor cell isolation kit” (Miltenyi Biotec, Auburn, Calif.) according to the manufacturer's recommendations. The purity of the CD34⁺ cells obtained ranged between 95% and 98%, based on Flow Cytometry evaluation.

Ex-vivo expansion of hematopoietic stem cells: The Mononuclear cells (MNC), obtained as described hereinabove, were plated in 24-well Costar Cell Clusters (Corning Inc., Corning, N. Y.) or seeded in Culture Bags (American Fluoroseal Corp.), with alpha minimal essential medium (a-MEM) supplemented with 10% fetal bovine serum (FBS, Biological Industries), at a concentration of about 10⁶ cells/ml. The purified CD34⁺ cells were similarly plated or seeded in the Culture Bags, at a concentration of about 10⁴ cells/ml. The media were supplemented with tetraethylpantamine (TEPA) chelator (obtained from Sigma) and/or with the following human recombinant cytokines (all obtained from Perpo Tech, Inc., Rocky Hill, N.J.): Thrombopoietin (TPO), 50 ng/ml; interleukin 6 (IL-6), 50 ng/ml; FLT-3 ligand, 50 ng/ml and a stem cell factor (SCF), 50 ng/ml; occasionally SCF was replaced by IL-3, 20 ng/ml. All cultures were incubated at 37° C. in an atmosphere of 5% CO₂ in air with extra humidity. At weekly intervals, the cell cultures were semi-depopulated and supplemented with fresh medium containing cytokines. Following different incubation periods, cells were harvested, stained with trypan blue and enumerated.

Morphological assessment: Morphological characterization of the resulting culture populations was accomplished on aliquots of cells deposited on glass slides via cytospin (Cytocentrifuge, Shandon, Runcorn, UK). Cells were fixed, stained with May-Grunwald/Giemsa stain and examined microscopically.

Surface antigen analysis: Cells were harvested, washed with a PBS solution containing 1% bovine sera albumin (BSA) and 0.1% sodium azide (Sigma), ind stained at 4° C. for 60 minutes with fluorescein isothiocyanate or phycoerythrin-conjugated antibodies (all from Immunoquality Products, the Netherlands). The cells were then washed with the same buffer and analyzed by FACS caliber or Facstarplus flow cytometers. Cells were passed at a rate of 1000 cells/second, using saline as the sheath fluid. A 488 nm argon laser beam served as the light source for excitation. Emission of ten thousand cells was measured using logarithmic amplification, and analyzed using CellQuest software.

Determination of CD34+ cells and subsets: CD34+ surface expression on short and long-term cultures initiated either with purified CD34+ cells or the entire MNC fraction was determined as follows: CD34+ cells were positively reselected (Miltenyi kit) and counted. Purity was confirmed by subsequent FACS and cell morphology analysis, as is described hereinabove.

Reselected CD34+ cell subsets were stained for the following combination of antigens: CD34PE/CD38FITC and CD34PE/38-, 33-, 14-, 15-, 3, 4, 61, 19 (Lin) FITC.

Cel Population Calculations:

FACS analysis results are given as percentage values of cells. Absolute numbers of subsets are calculated from the absolute number of CD34+ cells.

Determination of baseline levels of CD34⁺/CD38− and CD34⁺/Lin⁻ cells was conducted as follows: CD34+ cells were purified from 3 thawed cord blood units and stained for the above markers. The mean of these experiments was considered as the baseline value.

Total cell counts, numbers of CD34+ cells and subsets, and CFU numbers are presented as cumulative numbers, with the assumption that the cultures had not been passaged; i.e., the number of cells per ml were multiplied by the number of passages performed.

Assaying Colony Forming Unit (CFU) ability: Cells were cloned in semi-solid, methylcellulose-containing medium supplemented with 2 IU/ml erythropoietin (Eprex, Cilag AG Int., Switzerland), stem cell factor and IL-3, both at 20 ng/ml, and G-CSF and GM-CSF, both at 10 ng/ml (all from Perpo Tech). Cultures were incubated for 14 days at 37° C., 5% CO₂ in a humidified atmosphere.

Determination of LTC-CFUc values: The ability of the cultures to maintain self-renewal was measured by determination of the content of colony forming unit cells in the long and extended long-term cultures (LTC-CFUc), as described in the references hereinabove.

### Experimental Results

Mononuclear cells (MNC) were seeded in culture bags and were provided with nutrients and cytokines (50 ng/ml FLt3, IL-6, TPO and SCF) as described above. The MNC cultures were either treated or untreated (untreated controls) with various concentrations (5-10 μM) of TEPA chelator. The treated MNC cultures were supplemented with TEPA for only the first three weeks and from week three onward were topped with chelator-free media. The pre-purified CD34⁺ cultures were not supplemented with TEPA and served as positive controls. The cultures were analyzed weekly during a 12-week period for the number of cells, CFUc, CD34+ and CD34+CD38− cells. In order to precisely determine the CD34+ cell content, CD34+ cells were weekly reselected and enumerated from each of the experimental groups (treated and untreated MNC cultures) and the positive control (CD34+ cultures).

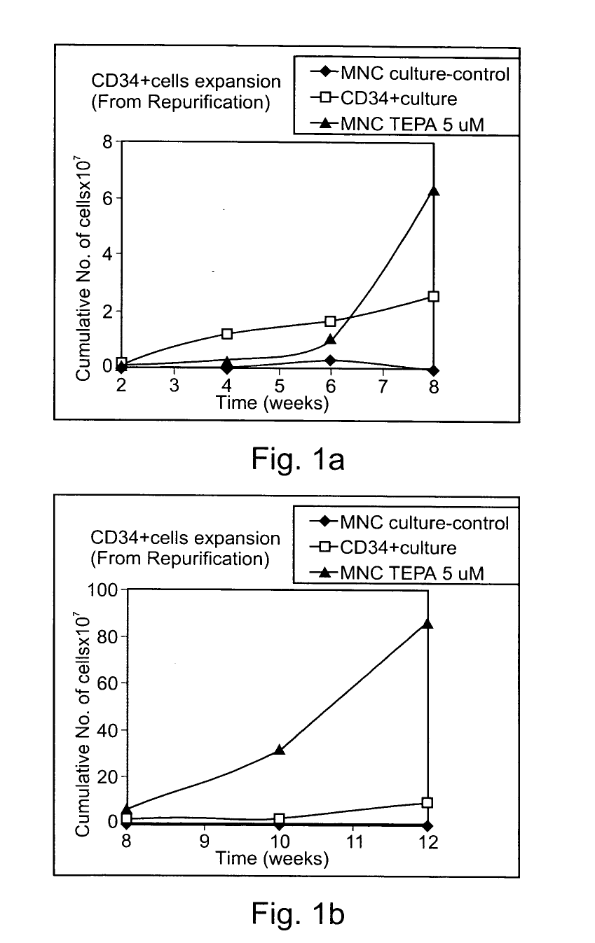

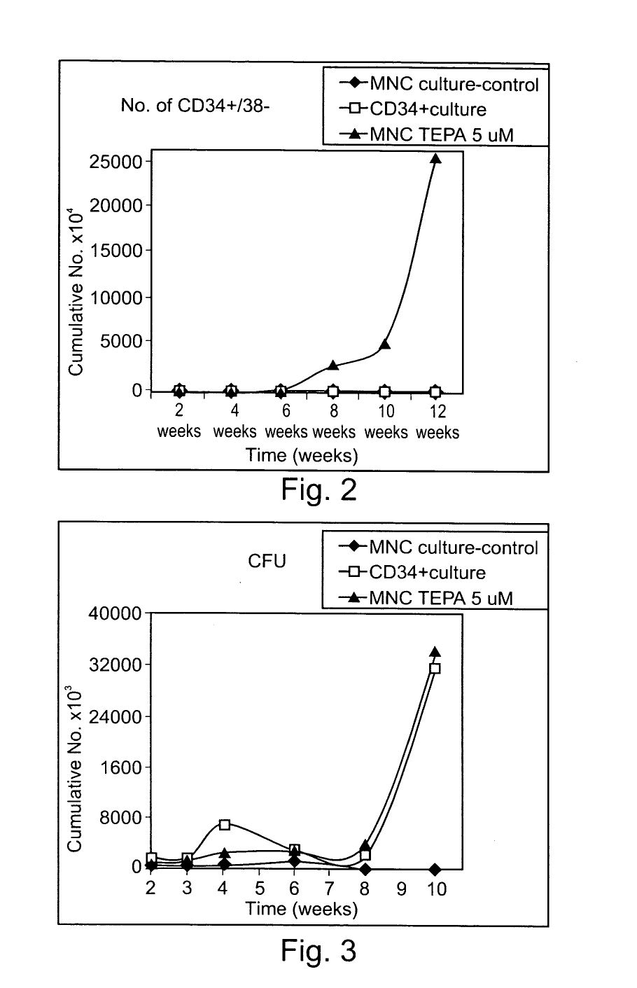

The results, illustrated in FIGS. 1a-b, 2 and 3, show that addition of TEPA chelator to non-purified MNC cultures, substantially and progressively increased the number of CD34⁺ cells, CD34⁺ colony-forming cells and CD34⁺CD38⁻ cells, over a 12-week period. Thus, in MNC cultures treated with TEPA, the cumulative number of CD34⁺ cells increased from a non-detectable level to over 8×10⁷ cells/ml, after 2 and 12 weeks, respectively (FIGS. 1a-b); the cumulative number of CD₃₄⁺CD₃₈⁻-cells increased from a non-detectable level to 2.5×10⁷ cells/ml, after 2 and 12 weeks, respectively (FIG. 2); and the number of CD34⁺ CFUs increased from a non-detectable level to 3.2×10⁷ cells/ml after 2 and 10 weeks, respectively (FIG. 3). On the other hand, when TEPA was not added to MNC cultures (untreated controls), no significant expansion of stem or progenitor cells was measured throughout the 1-week period. Furthermore, the stem and progenitor cells densities in the TEPA-treated MNC cultures, either equalized or surpassed the densities of stem and progenitor cells in pre-purified CD34⁺ cell cultures (not treated with TEPA, positive controls). Morphological analysis of cells derived from long-term and TEPA-treated MNC cultures, revealed a high proportion of non-differentiated cells, while most of the cells derived from long-term and MNC cultures not treated with TEPA, where fully differentiated.

The results described in this Example clearly show that stem and progenitor hematopoietic cells may be substantially expanded ex-vivo, continuously over at least 12 weeks period, in a culture of mixed (mononuclear fraction) blood cells, with no prior purification of CD34⁺ cells. The data also show that this effect resulted from supplementing the cells culture medium with TEPA chelator, only during the first three weeks of culturing.

These results indicate that short-term MNC cell cultures supplemented with TEPA in addition to cytokines, enabled tremendous expansion of CD34+ cells and stem/early progenitor cells (CD34+38−) as compared with minimal expansion of these cells obtained in MNC cultures treated only with cytokines. Comparison experiments demonstrated that expansion of CD34+ cells and its rare CD34+CD38-cell subset continue to occur in the extended long-term cultures and is much higher as compared with that obtained from cultures initiated with highly purified CD34+ cells. Therefore, the results may suggest that short-term treatment of MNC with TEPA potentiate the MNC cultures in a way that enables higher expansion of cells with extended self-renewal potential.

The results may also suggest that in addition to the regulatory effect on CD34+ cells and its early subsets, the chelator may also enable ex-vivo expansion a small subset of cells that are not co-purified with the CD34+ cell fraction. This subset of cells, which is probably in nature CD34−, may support superior expansion of CD34+ cells and its subsets during the extended long-term cultures.

Hence, this Example illustrates a substantial ex-vivo expansion of stem and progenitor cells in a mixed mononuclear cells culture. This novel procedure circumvents the need of the laborious and costly enrichment of stem cells prior so initiation of cultures, which is currently used in the art. Hence, the use of a copper chelator, such as TEPA, can substantially simplify, reduce cost and improve efficiency of procedures for an ex-vivo expansion of stem and/or progenitor cells.

### EXAMPLE 2

### The Effect of a Copper Chelate on the Ex-Vivo Expansion of Hema Topoietic Stem Cells of a Mononuclear Cells Culture

Copper-TEPA chelate was prepared as described, for example, in PCT/IL03/00062.

Mononuclear cells (MNC) were seeded in culture bags and were provided with nutrients and cytokines as described in Example 1 above. The mononuclear cell cultures were either untreated (control) or treated with Cu-TEPA chelate. The treated MNC cultures were supplemented with Copper-TEPA chelate for the first three weeks and from week three onward were topped with chelator-free media. All cultures were analyzed eight weeks after an 8-week period.

The results, presented in Table 1 below, show that addition of Copper-TEPA chelate to MNC cultures markedly increased the number of CD34⁺ cells, the proportion of CD34⁺ cells, and the number of CD34⁺CD38⁻ cells, after an eight weeks incubation period. Thus, the cumulative number of CD34⁺ cells per culture bag after incubation was 2.56×10⁶, 12.37×10⁶ or 32.85×10⁶, in the untreated cultures (cytokines only), 50 μM Copper-TEPA-treated and 100 μM Copper-TEPA-treated cultures, respectively. The cumulative number of CD₃₄⁺CD₃₈⁻ cells increased from 2.1×10⁵ in the untreated control culture (cytokines only) to 6.1×10⁵ in the Copper-TEPA (100 μM) treated culture.

TABLE 1

| | Number of | Portion of | Number of |
|---|---|---|---|
| | CD34 + cells | CD34 + cells | CD34/38 − cells |
| Treatment | (×10⁴) | (%) | (×10⁴) |
| Control | 256.0 | 0.2 | 21 |
| Cu-TEPA chelate | 1237.3 | 1.4 | — |
| 50 μM | | | |
| Cu-TEPA chelate | 3285.3 | 1.2 | 61 |
| 100 μM | | | |

The results described in this Example demonstrate that hematopoietic stem cells may be substantially expanded ex-vivo, over at least 8 weeks period, in a culture of mononuclear blood cells, with no prior purification of CD34+cells, in the presence of a copper chelate such as Copper-TEPA.

### EXAMPLE 3

### The Effect of a RAR Antagonist on the Ex-Vivo Expansion of Hema Topoietic Stem Cells of a Mononuclear Cells Culture

### Materials and Experimental Methods

The high-Affinity retinoic acid receptor (RAR) antagonist 4-[[4-(4-ethylphenyl)-2,2-dimethyl-(2H)-thiochomen-6-yl)]-benzoic acid, (AGN 194310) was synthesized according to the procedure described in PCT/IL03/00064.

Mononuclear cells fraction was collected and purified as described above in Example 1. MNC cultures were prepared and maintained as described above. AGN 194310 RAR antagonist was added to the tested cultures at concentrations ranging from 1×10⁻³-1×10⁻¹¹ M [or 410 μg/l to 4.1×10⁻⁵ μg/l]. The antagonist was added for a predetermined, limited period, for up to three weeks or continuously during the entire culture period.

The results, presented in Table 2, show that mononuclear cell fractions cultured in the presence of RAR antagonists and cytokines revealed a significant increase in the number of CD34+Lin− cells (78%, 24%) as quantitated by FACS analysis from a reselected, highly purified CD34+cell fraction, as compared with the control untreated MNC fractions, 2 and 5 weeks (respectively), after initial seeding. The MNC cells responded to the RAR antagonists and expanded an undifferentiated population, without prior purification of the CD34+ population. RAR antagonist treatment was sufficient to stimulate specific expansion of the stem/progenitor cell compartment, at 5 weeks post seeding. While control untreated MNCs had no detectable CD34+ population, RAR antagonist treated cultures revealed significant numbers of CD34+ cells, and those that were lineage marker deficient. Thus, any factors elaborated by the MNC culture cells that suppress CD34+ cell survival in control samples are insufficient to override the signal provided by the RAR antagonist to elaborate this compartment. 10

TABLE 2

| | |
|---|---|
| Cytokines | Cytokines + RAR |
| only | antagonist 10⁻⁶ M |

2 weeks

| | | |
|---|---|---|
| No of CD34 cells × 10⁴ | 176 | 169 |
| No of CD34⁺/Lin⁻ × 10⁴ | 1.76 | 132.5 |
| % CD34/Lin⁻ | 1 | 78.4 |

5 weeks

| | | |
|---|---|---|
| No of CD34 cells × 10⁴* | 0 | 985 |
| No of CD34⁺/Lin⁻ × 10⁴* | 0 | 237.8 |
| % CD34/Lin⁻ | 0 | 24.1 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior an to the present invention. The scope of the present invention and of the appended claims is not to be regarded as restricted or limited by or to any explicit or specific theory presented herein.

1-200. (cancelled).

201. A method of expanding an ex-vivo population of hematopoietic stem cells, while at the same time, substantially inhibiting differentiation of the hematopoietic stem cells ex-vivo, the method comprising
providing hematopoietic mononuclear cells;
culturing said mononuclear cells ex-vivo under conditions allowing for cell proliferation and, at the same time, culturing said cells under conditions selected from the group consisting of
conditions reducing expression and/or activity of CD38 in said mononuclear cells,
conditions reducing capacity of said hematopoietic mononuclear cells in responding to retinoic acid, retinoids and/or Vitamin D in said mononuclear cells,
conditions reducing capacity of said hematopoietic mononuclear cells in responding to signaling pathways involving the retinoic acid receptor, the retinoid X receptor and/or the Vitamin D receptor in said mononuclear cells;
culturing said mononuclear cells in the presence of nicotinamide, a nicotinamide analog, a nicotinamide or a nicotinamide analog derivative or a nicotinamide or a nicotinamide analog metabolite in said mononuclear cells;
conditions reducing an expression and/or activity of PI 3-kinase in said mononuclear cells; and
culturing said mononuclear cells in the presence of at least one copper chelator or chelate,
thereby expanding a population of said hematopoietic stem cells while at the same time substantially inhibiting differentiation of said hematopoietic stem cells ex-vivo.

202. A method of transplanting or implanting hematopoietic cells, the method comprising:
(a) obtaining hematopoietic mononuclear cells;
(b) culturing said mononuclear cells ex vivo for cell proliferation, wherein said culturing is performed in a condition selected from the group consisting of
reducing expression and/or activity of CD38,
reducing a capacity of said hematopoietic mononuclear cells in responding to retinoic acid, retinoids and/or Vitamin D,
reducing capacity of said hematopoietic mononuclear cells in responding to signaling pathways involving the retinoic acid receptor, the retinoid X receptor and/or the Vitamin D receptor;
the presence of nicotinamide, a nicotinamide analog, a nicotinamide or a nicotinamide analog derivative or a nicotinamide or a nicotinamide analog metabolite;
reducing an expression and/or activity of PI 3-kinase or
the presence of at least one copper chelator or chelate,
thereby expanding a population of said hematopoietic stem cells, while at the same time, substantially inhibiting differentiation of said hematopoietic stem cells ex-vivo; and
(c) transplanting or implanting said hematopoietic stem cells to a recipient.

203. The method of claim 202, wherein said donor and said recipient are a single individual.

204. A method of genetically modifying hematopoietic stem cells with an exogene comprising:
(a) obtaining hematopoietic mononuclear cells;
(b) culturing said mononuclear cells ex vivo for cell proliferation, wherein said culturing is performed in a condition selected from the group consisting of
conditions reducing expression and/or activity of CD38 in said mononuclear cells,
conditions reducing capacity of said hematopoietic mononuclear cells in responding to retinoic acid, retinoids and/or Vitamin D in said mononuclear cells,
conditions reducing capacity of said hematopoietic mononuclear cells in responding to signaling pathways involving the retinoic acid receptor, the retinoid X receptor and/or the Vitamin D receptor in said mononuclear cells;
culturing said mononuclear cells in the presence of nicotinamide, a nicotinamide analog, a nicotinamide or a nicotinamide analog derivative or a nicotinamide or a nicotinamide analog metabolite in said mononuclear cells;
conditions reducing an expression and/or activity of PI 3-kinase in said mononuclear cells; and
culturing said mononuclear cells in the presence of at least one copper chelator or chelate,
thereby expanding a population of said hematopoietic stem cells, while at the same time, substantially inhibiting differentiation of said hematopoietic stem cells ex-vivo; and
(c) genetically modifying said hematopoietic stem cells with the exogene.

205. The method of claim 204, wherein genetically modifying is effected by a vector which comprises the exogene.

206. The method of claim 205, wherein the vector is a viral vector or a nucleic acid vector.

207. A method of adoptive immunotherapy comprising:
(a) obtaining hematopoietic mononuclear cells from a recipient;
(b) culturing said mononuclear cells ex vivo for cell proliferation, wherein said culturing is performed in a condition selected from the group consisting of
conditions reducing expression and/or activity of CD38 in said mononuclear cells,
conditions reducing capacity of said hematopoietic mononuclear cells in responding to retinoic acid, retinoids and/or Vitamin D in said mononuclear cells,
conditions reducing capacity of said hematopoietic mononuclear cells in responding to signaling pathways involving the retinoic acid receptor, the retinoid X receptor and/or the Vitamin D receptor in said mononuclear cells;
culturing said mononuclear cells in the presence of nicotinamide, a nicotinamide analog, a nicotinamide or a nicotinamide analog derivative or a nicotinamide or a nicotinamide analog metabolite in said mononuclear cells;
conditions reducing an expression and/or activity of PI 3-kinase in said mononuclear cells; and
culturing said mononuclear cells in the presence of at least one copper chelator or chelate,
thereby expanding a population of said hematopoietic stem cells, while at the same time, substantially inhibiting differentiation of said hematopoietic stem cells; and
(c) transplanting said hematopoietic stem cells to the recipient.

208. A transplantable hematopoietic cell preparation comprising an expanded population of hematopoietic stem cells propagated ex-vivo from hematopoietic mononuclear cells in the presence of an effective amount of an agent,
wherein said agent has an activity selected from the group consisting of
reducing expression and/or activity of CD38 in said mononuclear cells,
reducing capacity of said hematopoietic mononuclear cells in responding to retinoic acid, retinoids and/or Vitamin D in said mononuclear cells,
reducing capacity of said hematopoietic mononuclear cells in responding to signaling pathways involving the retinoic acid receptor, the retinoid X receptor and/or the Vitamin D receptor in said mononuclear cells; and
reducing an expression and/or activity of PI 3-kinase in said mononuclear cells; or wherein said agent is
a copper chelator or chelate, or
nicotinamide, a nicotinamide analog, a nicotinamide or a nicotinamide analog derivative or a nicotinamide or a nicotinamide analog metabolite in said mononuclear cells;
while at the same time, substantially inhibiting differentiation of said hematopoietic stem cells, and a pharmaceutically acceptable carrier.

209. The method of claim 201, wherein said hematopoietic mononuclear cells are derived from a source selected from the group consisting of bone marrow, peripheral blood and neonatal umbilical cord blood.

210. The method of claim 201, wherein providing said hematopoietic mononuclear cells with said conditions for ex-vivo cell proliferation comprises providing said hematopoietic mononuclear cells with nutrients and with cytokines.

211. The method of claim 210, wherein said cytokines are early acting cytokines.

212. The method of claim 211, wherein said early acting cytokines are selected from the group consisting of stem cell factor, FLT3 ligand, interleukin-1, interleukin-2, interleukin-3, interleukin-6, interleukin-10, interleukin-12, tumor necrosis factor-a and thrombopoietin.

213. The method of claim 210, wherein said cytokines are late acting cytokines.

214. The method of claim 213, wherein said late acting cytokines are selected from the group consisting of granulocyte colony stimulating factor, granulocyte/macrophage colony stimulating factor, erythropoietin, FGF, EGF, NGF, VEGF, LIF, Hepatocyte growth factor and macrophage colony stimulating factor.

215. The method of claim 201, wherein providing said hematopoietic mononuclear cells with ex-vivo culture conditions for reducing said expression and/or said activity of CD38 is by providing said hematopoietic mononuclear cells with an agent that downregulates CD38 expression.

216. The transplantable hematopoietic cell preparation of claim 208, wherein said agent is an agent that downregulates CD38 expression.

217. The method of claim 215, wherein the agent that downregulates CD38 expression is selected from the group consisting of a retinoic acid receptor antagonist, a retinoid X receptor antagonist and a Vitamin D receptor antagonist.

218. The method of claim 215, wherein the agent that downregulates CD38 expression is an antagonist for reducing a capacity of said hematopoietic mononuclear cells in responding to retinoic acid, retinoid and/or Vitamin D.

219. The method of claim 215, wherein said agent that downregulates CD38 expression is a polynucleotide.

220. The method of claim 219, wherein the polynucleotide encodes an anti CD38, an anti retinoic acid receptor, an anti retinoid X receptor or an anti Vitamin D receptor intracellular antibody.

221. The method of claim 219, wherein the polynucleotide encodes an anti CD38, an anti retinoic acid receptor, an anti retinoid X receptor or an anti Vitamin D receptor antibody.

222. The method of claim 219, wherein said polynucleotide is a small interfering polynucleotide molecule directed to cause intracellular CD38, retinoic acid receptor, retinoid X receptor or Vitamin D receptor mRNA degradation.

223. The method of claim 222, wherein said small interfering polynucleotide molecule is selected from the group consisting of an RNAi molecule, an anti-sense molecule, a rybozyle molecule and a DNAzyme molecule.

224. The method of claim 215, wherein said agent that downregulates CD38 expression is an agent that downregulates PI 3-kinase expression.

225. The method of claim 224, wherein said agent that downregulates PI 3-kinase expression is a polynucleotide.

226. The method of claim 224, wherein agent that downregulates PI 3-kinase expression is an intracellular antibody.

227. The method of claim 225, wherein said polynucleotide is a small interfering polynucleotide molecule directed to cause intracellular PI 3-kinase mRNA or gene degradation.

228. The method of claim 227, wherein said small interfering polynucleotide molecule is selected from the group consisting of an RNAi molecule, an anti-sense molecule, a rybozyme molecule and a DNAzyme molecule.

229. The method of claim 215, wherein said agent that downregulates CD38 expression is an agent that inhibits PI 3-kinase activity.

230. The method of claim 229, wherein said agent that inhibits PI 3-kinase activity is selected from the group consisting of wortmannin and LY294002

231. The method of claim 201, wherein providing said hematopoietic mononuclear cells with ex-vivo culture conditions for reducing said expression and/or said activity of CD38 is by providing said hematopoietic mononuclear cells with an agent that inhibits CD38 activity.

232. The transplantable hematopoietic cell preparation of claim 208, wherein said agent is an agent that inhibits CD38 activity.

233. The method of claim 232, wherein said agent that inhibits CD38 activity is nicotinamide, a nicotinamide analog, a nicotinamide or a nicotinamide analog derivative or a nicotinamide or a nicotinamide analog metabolite.

234. The method of claim 233, wherein said nicotinamide analog is selected from the group consisting of benzamide, nicotinethioamide, nicotinic acid and α-amino-3-indolepropionic acid.

235. The method of claim 201, wherein providing said hematopoietic mononuclear cells with ex-vivo culture conditions for reducing said expression and/or said activity of CD38 is by providing said hematopoietic mononuclear cells with an agent that inhibits PI 3-kinase activity.

236. The transplantable hematopoietic cell preparation of claim 208, wherein said agent is an agent that inhibits PI 3-kinase activity.

237. The method of claim 236, wherein said agent that inhibits PI 3-kinase activity is selected from the group consisting of wortmannin and LY294002.

238. The method of claim 201, wherein said hematopoietic mononuclear cells are not enriched prior to culturing ex-vivo under conditions allowing for cell proliferation.

239. The method of claim 201, wherein said hematopoietic cells comprise a major fraction of hematopoietic committed cells and a minor fraction of hematopoietic stem and progenitor cells.

240. An assay for determining whether a transition metal chelate or chelator causes substantial inhibition or induction of differentiation of hematopoietic stem cells, the assay comprising:
culturing hematopoietic mononuclear cells in the presence of the transition metal chelate or chelator and monitoring differentiation of said hematopoietic stem cells, wherein if differentiation is increased as is compared to non-treated hematopoietic mononuclear cells, said transition metal chelate induces differentiation, whereas if differentiation is decreased as is compared to non-treated hematopoietic mononuclear cells, or if differentiation is absent altogether, said transition metal chelate inhibits differentiation.

241. An assay for identifying an effective hematopoietic stem cell expansion agent, the assay comprising culturing hematopoietic mononuclear cells in the presence of a compound selected from the group consisting of
a retinoic acid receptor antagonist;
retinoid X receptor antagonist;
vitamin D receptor antagonist;
agent that inhibits PI 3-kinase activity; and
a nicotinamide analog, a nicotinamide or a nicotinamide analog derivative or a nicotinamide or a nicotinamide analog metabolite,
and monitoring expansion of said hematopoietic stem cells, wherein if increased expansion and decreased differentiation of said hematopoietic stem cells occurs, as compared to non-treated hematopoietic mononuclear cells, the compound is an effective hematopoietic stem cell expansion agent.

242. A hematopoietic stem cells collection/culturing bag supplemented with an effective amount of a compound selected from the group consisting of
a retinoic acid receptor antagonist, a retinoid X receptor antagonist and/or a Vitamin D receptor antagonist,
nicotinamide, a nicotinamide analog, a nicotinamide or a nicotinamide analog derivative or a nicotinamide or a nicotinamide analog metabolite; or
an agent that inhibits PI 3-kinase activity,
which substantially inhibits cell differentiation of a hematopoietic stem cells fraction of hematopoietic mononuclear cells.

243. An ex-vivo expanded population of hematopoietic stem cells, obtained by the method of claim 1.
